(19) United States
(12) Patent Application Publication
(10) Pub. No.: US 2021/0087560 A1
(43) Pub. Date: Mar. 25, 2021

(54) OLIGODENDROCYTE-SPECIFIC PROMOTER, MIRNA SPECIFIC TO PLP1 GENE,VECTOR INCLUDING SAID PROMOTER AND/OR MIRNA, AND PHARMACEUTICAL COMPOSITION INCLUDING SAID VECTOR

(71) Applicants: NATIONAL CENTER OF NEUROLOGY AND PSYCHIATRY, Kodaira-shi, Tokyo (JP); NIPPON MEDICAL SCHOOL FOUNDATION, Tokyo (JP); DAIICHI SANKYO COMPANY, LIMITED, Tokyo (JP)

(72) Inventors: Ken Inoue, Kodaira-shi, Tokyo (JP); Heng Li, Kodaira-shi, Tokyo (JP); Takashi Okada, Bunkyo-ku, Tokyo (JP); Yu Ohki, Chuo-ku, Tokyo (JP); Makoto Koizumi, Chuo-ku, Tokyo (JP)

(73) Assignees: NATIONAL CENTER OF NEUROLOGY AND PSYCHIATRY, Kodaira-shi, Tokyo (JP); NIPPON MEDICAL SCHOOL FOUNDATION, Tokyo (JP); DAIICHI SANKYO COMPANY, LIMITED, Tokyo (JP)

(21) Appl. No.: 16/967,483

(22) PCT Filed: Feb. 6, 2019
(86) PCT No.: PCT/JP2019/004227

§ 371 Date: Aug. 5, 2020

(30) Foreign Application Priority Data
Feb. 7, 2018 (JP) 2018-019950

(51) Int. Cl. C12N 15/113; C12N 15/86

(52) U.S. Cl. CPC C12N 15/113; C12N 15/86; C12N 2320/32; C12N 2310/141; C12N 2750/14143

(57) ABSTRACT

An object of the present invention is to provide a vector capable of oligodendrocyte-specifically suppressing expression of the PLP1 gene for treating PMD caused by abnormality of the PLP1 gene, and a promoter and miRNA therefor, and a pharmaceutical composition comprising the vector. The oligodendrocyte-specific promoter of the present invention comprises a nucleic acid having a sequence identity of at least 90% to a nucleotide sequence set forth in SEQ ID NO: 1. The miRNA of the present invention specific to the PLP1 gene comprises a pair of nucleotide sequences consisting of a specific antisense sequence and sense sequence.

### TECHNICAL FIELD

The present invention relates to an oligodendrocyte-specific promoter, in particular, to an oligodendrocyte-specific promoter comprising a nucleic acid having a sequence identity of at least 90% to a nucleotide sequence set forth in SEQ ID NO: 1. The present invention also relates to an miRNA specific to a PLP1 gene. Further, the present invention relates to a vector comprising the promoter and/or the miRNA, and a pharmaceutical composition comprising the vector.

### BACKGROUND ART

Hypomyelinating leukodystrophies is a collective term for brain diseases in children caused by poor development of the brain white matter, and currently 11 diseases have been known. A representative disease among them is Pelizaeus-Merzbacher disease (PMD). PMD is a rare intractable disease that is characterized in that myelination of the central nervous system is disordered and causes developmental failure of motor functions and neurological symptoms immediately after birth. The estimated incidence in Japan is 1.45 individuals per 100000 newborn male infants. Currently, there is no fundamental cure.

Myelin is generated by oligodendrocytes (oligodendroglia), one of glial cells, in the brain, and has a multilayer structure of insulating phospholipid present around the axon of a neuron. A primary protein constituting myelin is proteolipid protein (PLP), Myelin basic protein (MBP), human 2′,3′-cyclic nucleotide phosphodiesterase (CNP), and so on are also known as other constituents. PMD is caused by abnormalities of a gene for proteolipid protein (PLP1 gene). The most frequent mutation among such abnormalities is PLP1 duplications (about 60%). PLP1 duplication leads to overexpression of the PLP1 gene, thereby normalization of its expression level is expected to provide therapeutic effect.

Non Patent Literature 1 disclosed expression of adeno-associated virus (AAV)-mediated green fluorescent protein (GFP) driven by myelin basic protein (MBP) promoter or glial fibrillary acidic protein (GFAP) promoter in the brain of mice under development, and reported that the expression of GFAP promoter-driven GFP was highly specific to astrocytes after injection of a vector into the brain of neonatal mice and adult mice. Non Patent Literature 1 also reported the selectivity of the MBP promoter for oligodendrocytes was poor after AAV delivery to right after birth, but was superior after injection of a vector 10 days after birth. This document suggested that direct injection of AAV driven by a cell type-specific promoter into the developed brain after birth generates targeted long-term transgene expression in glial cells.

Non Patent Literature 2 disclosed development of target gene therapy for oligodendrocytes in Pelizaeus-Merzbacher-like disease, and reported that a GJC2/Cx47 gene was inserted into the downstream of a myelin basic protein promoter, and administered to 10-day-old mice via single intracerebral injection into the internal capsule with an adeno-associated virus (AAV.MBP.Cx47myc) vector.

Each of Non Patent Literatures 1 and 2 discloses gene therapy and a promoter therefor with use of an AAV vector, but did not describe at all gene therapy for abnormality of the PLP1 gene and a promoter or miRNA therefor.

### CITATION LIST

### Non Patent Literature

Non Patent Literature 1: von Jonquieres G et al., Glial promoter selectivity following AAV-delivery to the immature brain. PLoS One. 2013 Jun. 14; 8(6): e65646.
Non Patent Literature 2: Georgiou E et al., Gene therapy targeting oligodendrocytes provides therapeutic benefit in a leukodystrophy model. Brain. 2017 Mar. 1; 140(3): 599-616.

### SUMMARY OF INVENTION

### Technical Problem

An object of the present invention is to provide a vector capable of oligodendrocyte-specifically suppressing expression of the PLP1 gene for treating PMD caused by abnormality of the PLP1 gene, and a promoter and miRNA therefor, and a pharmaceutical composition comprising the vector.

### Solution to Problem

The present inventors found that a human 2′,3′-cyclic nucleotide phosphodiesterase (CNP) promoter having a sequence set forth in SEQ ID NO: 1 can drive gene expression in an oligodendrocyte-specific and highly efficient manner, and that expression of the PLP1 gene is successfully suppressed with an AAV vector comprising the promoter and a PLP1 gene-specific miRNA operably linked to the downstream of the promoter, thus completing the present invention.

Specifically, the present invention relates to the followings.
[1] An oligodendrocyte-specific promoter comprising a nucleic acid having a sequence identity of at least 90% to a nucleotide sequence set forth in SEQ ID NO: 1.
[2] The promoter according to [1], comprising a nucleic acid having the nucleotide sequence set forth in SEQ ID NO: 1.
[3] An oligodendrocyte-specific vector comprising the promoter according to [1] or [2].
[4] The vector according to [3], further comprising an miRNA sequence specific to a human PLP1 gene operably linked to the promoter.
[5] An miRNA comprising a pair of nucleotide sequences consisting of an antisense sequence and a sense sequence and being specific to a PLP1 gene, wherein the pair of nucleotide sequences is selected from the group consisting of pairs of an antisense sequence set forth in a left column of Table 1 and a sense sequence set forth in the just right of the antisense sequence in a right column of the table.
[6] The miRNA according to [5], comprising a nucleotide sequence selected from the group consisting of SEQ ID NOs: 2 to 7 and 24 to 51.
[7] The miRNA according to [5], comprising a nucleotide sequence selected from the group consisting of SEQ ID NOs: 2 to 5, 7, 24, 26, 29 to 32, 35, 36, 42, and 51.
[8] The miRNA according to [5], comprising a nucleotide sequence selected from the group consisting of SEQ ID NOs: 2, 29, 31, and 32.
[9] A vector comprising a sequence of the miRNA according to any one of [5] to [8].
[10] The vector according to [9], wherein the sequence of the miRNA is operably linked to an oligodendrocyte-specific promoter.
[11] The vector according to [10], wherein the oligodendrocyte-specific promoter is the promoter according to [1] or [2].
[12] The vector according to any one of [3], [4], and [9] to [11], wherein the vector is an adeno-associated virus (AAV) vector.
[13] A pharmaceutical composition comprising the vector according to any one of [3], [4], and [9] to [12].
[14] The pharmaceutical composition according to [13], wherein the pharmaceutical composition is a pharmaceutical composition for treating a disease associated with abnormality in a PLP1 gene.
[15] The pharmaceutical composition according to [14], wherein the disease is Pelizaeus-Merzbacher disease, spastic paraplegia type 2, or multiple sclerosis.
[16] The pharmaceutical composition according to [14], wherein the disease is Pelizaeus-Merzbacher disease.
[17] The pharmaceutical composition according to [13], wherein the pharmaceutical composition is a PLP1 gene expression suppressor which suppresses expression of the PLP1 gene.
[18] A method for treating a disease associated with abnormality of a PLP1 gene, the method comprising administering an effective amount of the vector according to any one of [3], [4], and [9] to [12] to a patient in need of treatment.
[19] The method according to [18], wherein the disease is Pelizaeus-Merzbacher disease, spastic paraplegia type 2, or multiple sclerosis.
[20] The method according to [18], wherein the disease is Pelizaeus-Merzbacher disease.
[21] A method for suppressing expression of a PLP1 gene, the method comprising administering an effective amount of the vector according to any one of [3], [4], and [9] to [12] to a patient in need of treatment.
[22] The vector according to any one of [3], [4], and [9] to [12] for use in treatment of a disease associated with abnormality of a PLP1 gene.
[23] The vector according to [22], wherein the disease is Pelizaeus-Merzbacher disease, spastic paraplegia type 2, or multiple sclerosis.
[24] The vector according to [22], wherein the disease is Pelizaeus-Merzbacher disease.

### Advantageous Effects of Invention

The AAV vector comprising the promoter of the present invention is capable of expressing a gene in an oligodendrocyte-specific and highly efficient manner, and moreover successfully suppresses expression of the PLP1 gene through inclusion of an miRNA sequence specific to the human PLP1 gene operably linked to the promoter, and thus can serve as a therapeutic drug for PMD caused by PLP1 duplication, etc.

### BRIEF DESCRIPTION OF DRAWINGS

FIG. 1 shows a schematic diagram of an AAV vector of Example 1.

FIG. 2 shows photographs representing results of immunofluorescence staining on specificity of AAV-expressing cells in Example 2.

FIG. 3 shows a graph representing results of site-by-site measurement of suppression of expression (knockdown) in terms of PLP1 gene expression levels on the basis of fluorescence intensity generated by PLP1 immunofluorescence staining in Example 3.

FIG. 4 shows graphs representing results of measurement of suppression of expression (knockdown) in terms of PLP1 gene expression levels through quantitative PCR in Example 4. (A) and (B) show relative expression levels of the PLP1 gene and relative expression levels of the Olig2 gene, respectively.

FIG. 5 shows photographs representing results of histological analysis through immunostaining after administration to PLP1-Tg mice in Example 5.

FIG. 6 shows photographs representing results of micromorphological analysis with an electron microscope after administration to PLP1-Tg mice in Example 5.

FIG. 7 shows a graph representing life-prolonging effect provided by administration to PLP1-Tg mice in Example 5.

FIG. 8 shows a graph representing body weight gain effect provided by administration to PLP1-Tg mice in Example 5.

FIG. 9 shows a graph representing results of measurement of suppression of expression (knockdown) in terms of PLP1 gene expression levels through quantitative PCR in Example 6.

FIG. 10 shows a graph representing results of measurement of suppression of expression (knockdown) in terms of PLP1 gene expression levels through quantitative PCR in Example 7.

FIG. 11 shows a graph representing results of measurement of suppression of expression (knockdown) in terms of PLP1 gene expression levels through quantitative PCR in Example 8.

FIG. 12 shows a graph representing results of measurement of suppression of expression (knockdown) in terms of PLP1 gene expression levels through quantitative PCR in Example 9.

FIG. 13 shows a graph representing results of site-by-site measurement of suppression of expression (knockdown) in terms of PLP1 gene expression levels on the basis of fluorescence intensity generated by PLP1 immunofluorescence staining in Example 10.

### DESCRIPTION OF EMBODIMENTS

[Promoter]

The promoter of the present embodiment is an oligodendrocyte-specific promoter comprising a nucleic acid having a sequence identity of at least 90%, preferably having a sequence identity of at least 95%, more preferably having a sequence identity of at least 98%, to a nucleotide sequence set forth in SEQ ID NO: 1, particularly preferably comprising a nucleic acid having the nucleotide sequence set forth in SEQ ID NO: 1. In addition to the nucleic acid having a sequence identity of at least 90% to the nucleotide sequence set forth in SEQ ID NO: 1, the promoter may include in an end of the promoter nucleotide sequences of sites for cloning into vectors such as restriction enzyme sites and an att sequence for Gateway cloning.

The nucleic acid having the nucleotide sequence set forth in SEQ ID NO: 1 is a human CNP promoter, and derived from chr17:40,118,309-40,120,101 of the human genome reference sequence (GRCh37/hg19). The human CNP gene is a gene expressed specifically in oligodendrocytes in the brain, and the specificity is inferred to be determined by the promoter sequence. The present inventors found that the human CNP promoter sequence set forth in SEQ ID NO: 1 is capable of oligodendrocyte-specifically expressing genes other than the CNP gene that are disposed in the downstream of the promoter.

The promoter of the present embodiment is a promoter that specifically operates in oligodendrocytes, whereby genes operably linked to the downstream of the promoter are expressed specifically in oligodendrocytes. The promoter of the present embodiment can be obtained through a gene engineering approach. For example, a gene for the human CNP promoter is obtained from a human genome, and some nucleotides are deleted, inserted, or substituted to obtain a promoter having a desired nucleotide sequence. Alternatively, a promoter having a desired nucleotide sequence can be obtained through partial or complete synthesis.

[miRNA]

An miRNA typically includes an antisense sequence and a sense sequence, and can form a hairpin structure, thereby processed into a mature miRNA. The antisense strand in the mature miRNA bonds to a target mRNA to decompose the mRNA or cause translational repression, thereby suppressing expression of the target gene. The miRNA of the present embodiment is an miRNA specific to the PLP1 gene, in particular, to the human PLP1 gene, includes an antisense sequence and a sense sequence, and is capable of suppressing expression of the PLP1 gene.

It is preferable that a 5′ flanking sequence, an antisense sequence, a stem-loop sequence, a sense sequence, and a 3′ flanking sequence be disposed in the order from the 5′ side in the miRNA of the present embodiment. The configuration is required to be such that the 5′ flanking sequence has a length of 25 to 100 nucleotides, the stem-loop sequence has a length of 4 to 30 nucleotides, and the 3′ flanking sequence has a length of 25 to 100 nucleotides.

Each flanking sequence needs to be a flanking sequence known to be generally expressed in oligodendrocytes, and, for example, a 5′ flanking sequence and 3′ flanking sequence that are known to be sequences derived from miR155 can be used. In addition to the 5′ flanking sequence and 3′ flanking sequence derived from miR155, a 5′ flanking sequence and 3′ flanking sequence of any of miR-138, miR-219, miR-9, miR-23a, miR-388, and miR-297c, which are known to be expressed in oligodendrocytes, can be used.

The miRNA sequence specific to the human PLP1 gene can be designed, for example, by using software such as BLOCK-iT™ RNAi Designer (Thermo Fisher Scientific, USA), and, for example, the following human PLP1-miRNA candidate nucleotide sequences can be used.

Human PLP1-miRNA candidate nucleotide sequence 1
(SEQ ID NO: 2):
5′-
AAAGGAAGAAGAAAGAGGCAGGTTTTGGCCACTGACTGACC
TGCCTCTCTTCTTCCTTT-3′
Human PLP1-miRNA candidate nucleotide sequence 2
(SEQ ID NO: 3):
5′-
AACACCAGGAGCCACACAACGGTTTTGGCCACTGACTGACCG
TTGTGTCTCCTGGTGTT-3′
Human PLP1-miRNA candidate nucleotide sequence 3
(SEQ ID NO: 4):
5′-
TTCCATGGGAGAACACCATACGTTTTGGCCACTGACTGACGTA
TGGTGCTCCCATGGAA-3′
Human PLP1-miRNA candidate nucleotide sequence 4
(SEQ ID NO: 5):
5′-
TGAGCAGGGAAACCAGTGTAGGTTTTGGCCACTGACTGACCT
ACACTGTTCCCTGCTCA-3′
Human PLP1-miRNA candidate nucleotide sequence 5
(SEQ ID NO: 6):
5′-
AGGGCTTTCTGATTGACAGCCGTTTTGGCCACTGACTGACGG
CTGTCACAGAAAGCCCT-3′
Human PLP1-miRNA candidate nucleotide sequence 6
(SEQ ID NO: 7):
5′-
ACCCCAAAGAAACACAATCCAGTTTTGGCCACTGACTGACtgg
attgtttctttggggt-3′
Human PLP1-miRNA candidate nucleotide sequence 7
(SEQ ID NO: 24):
5′-
ACAAATGCAGCAATAAACAGGGTTTTGGCCACTGACTGACCC
TGTTTAGCTGCATTTGT-3′
Human PLP1-miRNA candidate nucleotide sequence 8
(SEQ ID NO: 25):
5′-
AATAGACTGGCAGGTGGTCCAGTTTTGGCCACTGACTGACTG
GACCACGCCAGTCTATT-3′
Human PLP1-miRNA candidate nucleotide sequence 9
(SEQ ID NO: 26):
5′-
AAAGAATGAGCTTGATGTTGGGTTTTGGCCACTGACTGACCC
AACATCGCTCATTCTTT-3′
Human PLP1-miRNA candidate nucleotide sequence 10
(SEQ ID NO: 27):
5′-
AGATACTCATAGTCTTGGTAGGTTTTGGCCACTGACTGACCTA
CCAAGTATGAGTATCT-3′
Human PLP1-miRNA candidate nucleotide sequence 11
(SEQ ID NO: 28):
5′-
AAGCCCATGTCTTTGGGACTCGTTTTGGCCACTGACTGACGA
GTCCCAGACATGGGCTT-3′

Alternatively, the miRNA sequence specific to the human PLP1 gene can be designed in such a manner that artificial siRNAs are designed as described in Examples, and an optimum sequence is selected from the siRNAs to design the miRNA.

The miRNA of the present embodiment comprises a pair of nucleotide sequences consisting of an antisense sequence and a sense sequence, and the pair of nucleotide sequences may be selected from the group consisting of pairs of an antisense sequence set forth in a left column of Table 1 and a sense sequence set forth in the just right of the antisense sequence in a right column of the table.

TABLE 1
Antisense and sense sequences for miRNA designed for human
PLP1 gene

| Antisense sequence | Sense sequence |
|---|---|
| AAAGGAAGAAGAAAGAGGCAG (SEQ ID NO: | CTGCCTCTCTTCTTCCTTT (SEQ ID NO: 53) |
| 52) | |
| AACACCAGGAGCCACACAACG (SEQ ID NO: | CGTTGTGTCTCCTGGTGTT (SEQ ID NO: 55) |
| 54) | |
| TTCCATGGGAGAACACCATAC (SEQ ID NO: | GTATGGTGCTCCCATGGAA (SEQ ID NO: 57) |
| 56) | |
| TGAGCAGGGAAACCAGTGTAG (SEQ ID NO: | CTACACTGTTCCCTGCTCA (SEQ ID NO: 59) |
| 58) | |
| AGGGCTTTCTGATTGACAGCC (SEQ ID NO: | GGCTGTCACAGAAAGCCCT (SEQ ID NO: 61) |
| 60) | |
| ACCCCAAAGAAACACAATCCA (SEQ ID NO: | TGGATTGTTTCTTTGGGGT (SEQ ID NO: 63) |
| 62) | |
| ACAAATGCAGCAATAAACAGG (SEQ ID NO: | CCTGTTTAGCTGCATTTGT (SEQ ID NO: 65) |
| 64) | |
| AATAGACTGGCAGGTGGTCCA (SEQ ID NO: | TGGACCACGCCAGTCTATT (SEQ ID NO: 67) |
| 66) | |
| AAAGAATGAGCTTGATGTTGG (SEQ ID NO: | CCAACATCGCTCATTCTTT (SEQ ID NO: 69) |
| 68) | |
| AGATACTCATAGTCTTGGTAG (SEQ ID NO: | CTACCAAGTATGAGTATCT (SEQ ID NO: 71) |
| 70) | |
| AGAAACACAATCCAGTGGCCA (SEQ ID NO: | TGGCCACTATTGTGTTTCT (SEQ ID NO: 73) |
| 72) | |
| AAATAGGTCTCAATTAGCTTT (SEQ ID NO: | AAAGCTAAGAGACCTATTT (SEQ ID NO: 75) |
| 74) | |
| AAGAAACACAATCCAGTGGCC (SEQ ID NO: | GGCCACTGTTGTGTTTCTT (SEQ ID NO: 77) |
| 76) | |
| TAAACAGGTGGAAGGTCATTT (SEQ ID NO: | AAATGACCCCACCTGTTTA (SEQ ID NO: 79) |
| 78) | |
| TTGTAGTCGCCAAAGATCTGC (SEQ ID NO: | GCAGATCTGGCGACTACAA (SEQ ID NO: 81) |
| 80) | |
| AATTAGAGCCTCCATTCCTTT (SEQ ID NO: | AAAGGAATAGGCTCTAATT (SEQ ID NO: 83) |
| 82) | |
| TTAAGGACGGCAAAGTTGTAA (SEQ ID NO: | TTACAACTGCCGTCCTTAA (SEQ ID NO: 85) |
| 84) | |
| TTTAAGGACGGCAAAGTTGTA (SEQ ID NO: | TACAACTTCCGTCCTTAAA (SEQ ID NO: 87) |
| 86) | |
| ATGTCTTTGGGACTCTGACTC (SEQ ID NO: | GAGTCAGACCCAAAGACAT (SEQ ID NO: 89) |
| 88) | |
| TATCTATCCTGTGTCTACCAG (SEQ ID NO: | CTGGTAGACAGGATAGATA (SEQ ID NO: 91) |
| 90) | |
| AAATTACTTTCTGATCCTCAG (SEQ ID NO: | CTGAGGATGAAAGTAATTT (SEQ ID NO: 93) |
| 92) | |
| TCTAACAAGCCCATGTCTTTG (SEQ ID NO: | CAAAGACAGGCTTGTTAGA (SEQ ID NO: 95) |
| 94) | |
| AATTACTTTCTGATCCTCAGG (SEQ ID NO: | CCTGAGGAAGAAAGTAATT (SEQ ID NO: 97) |
| 96) | |
| AGTAAATGTACACAGGCACAG (SEQ ID NO: | CTGTGCCTGTACATTTACT (SEQ ID NO: 99) |
| 98) | |
| TAAGTAAGGTTGGCTGAGTTA (SEQ ID NO: | TAACTCAGAACCTTACTTA (SEQ ID NO: |
| 100) | 101) |
| TTCTGTGGGTGAAAGATCCTT (SEQ ID NO: | AAGGATCTCACCCACAGAA (SEQ ID NO: |
| 102) | 103) |
| AGAAGATGCTGACAACACCCT (SEQ ID NO: | AGGGTGTTCAGCATCTTCT (SEQ ID NO: |
| 104) | 105) |
| AATTGTAGCCGGCTGGCTAGT (SEQ ID NO: | ACTAGCCACGGCTACAATT (SEQ ID NO: |
| 106) | 107) |
| AGATTTGGGCAAACGCTCTTA (SEQ ID NO: | TAAGAGCGTGCCCAAATCT (SEQ ID NO: |
| 108) | 109) |
| ATTCTACGCTCCCTTATGCTG (SEQ ID NO: | CAGCATAAGAGCGTAGAAT (SEQ ID NO: |
| 110) | 111) |
| TGGTAATAGAGAGACCAGAAT (SEQ ID NO: | ATTCTGGTCTCTATTACCA (SEQ ID NO: |
| 112) | 113) |
| TATAGATGGCAAGAGGACCAA (SEQ ID NO: | TTGGTCCTTGCCATCTATA (SEQ ID NO: |
| 114) | 115) |
| AAACCAGTGTAGCTGCAGCCC (SEQ ID NO: | GGGCTGCATACACTGGTTT (SEQ ID NO: |
| 116) | 117) |

The miRNA of the present embodiment preferably includes any of the following pairs of nucleotide sequences.
A pair of nucleotide sequences consisting of SEQ ID NO: 52 as an antisense sequence and SEQ ID NO: 53 as a sense sequence
A pair of nucleotide sequences consisting of SEQ ID NO: 54 as an antisense sequence and SEQ ID NO: 55 as a sense sequence
A pair of nucleotide sequences consisting of SEQ ID NO: 56 as an antisense sequence and SEQ ID NO: 57 as a sense sequence
A pair of nucleotide sequences consisting of SEQ ID NO: 58 as an antisense sequence and SEQ ID NO: 59 as a sense sequence
A pair of nucleotide sequences consisting of SEQ ID NO: 62 as an antisense sequence and SEQ ID NO: 63 as a sense sequence
A pair of nucleotide sequences consisting of SEQ ID NO: 64 as an antisense sequence and SEQ ID NO: 65 as a sense sequence
A pair of nucleotide sequences consisting of SEQ ID NO: 68 as an antisense sequence and SEQ ID NO: 69 as a sense sequence
A pair of nucleotide sequences consisting of SEQ ID NO: 72 as an antisense sequence and SEQ ID NO: 73 as a sense sequence
A pair of nucleotide sequences consisting of SEQ ID NO: 76 as an antisense sequence and SEQ ID NO: 77 as a sense sequence
A pair of nucleotide sequences consisting of SEQ ID NO: 78 as an antisense sequence and SEQ ID NO: 79 as a sense sequence
A pair of nucleotide sequences consisting of SEQ ID NO: 84 as an antisense sequence and SEQ ID NO: 85 as a sense sequence
A pair of nucleotide sequences consisting of SEQ ID NO: 86 as an antisense sequence and SEQ ID NO: 87 as a sense sequence
A pair of nucleotide sequences consisting of SEQ ID NO: 98 as an antisense sequence and SEQ ID NO: 99 as a sense sequence
A pair of nucleotide sequences consisting of SEQ ID NO: 116 as an antisense sequence and SEQ ID NO: 117 as a sense sequence

The miRNA of the present embodiment more preferably includes any of the following pairs of nucleotide sequences.
A pair of nucleotide sequences consisting of SEQ ID NO: 52 as an antisense sequence and SEQ ID NO: 53 as a sense sequence
A pair of nucleotide sequences consisting of SEQ ID NO: 72 as an antisense sequence and SEQ ID NO: 73 as a sense sequence
A pair of nucleotide sequences consisting of SEQ ID NO: 76 as an antisense sequence and SEQ ID NO: 77 as a sense sequence
A pair of nucleotide sequences consisting of SEQ ID NO: 78 as an antisense sequence and SEQ ID NO: 79 as a sense sequence

Moreover, the miRNA of the present embodiment sequence may have a nucleotide sequence selected from the group consisting of SEQ ID NOs: 29 to 51 set forth in Table 2.

TABLE 2
miRNA Sequences designed for human PLP1 gene

| SEQ ID | |
|---|---|
| NO: | pre-miRNA Sequence |
| 29 | AGAAACACAATCCAGTGGCCAGTTTTGGCCACTGACTGACTGGCCACTATTGTGTTTCT |
| 30 | AAATAGGTCTCAATTAGCTTTGTTTTGGCCACTGACTGACAAAGCTAAGAGACCTATTT |
| 31 | AAGAAACACAATCCAGTGGCCGTTTTGGCCACTGACTGACGGCCACTGTTGTGTTTCTT |
| 32 | TAAACAGGTGGAAGGTCATTTGTTTTGGCCACTGACTGACAAATGACCCCACCTGTTTA |
| 33 | TTGTAGTCGCCAAAGATCTGCGTTTTGGCCACTGACTGACGCAGATCTGGCGACTACAA |
| 34 | AATTAGAGCCTCCATTCCTTTGTTTTGGCCACTGACTGACAAAGGAATAGGCTCTAATT |
| 35 | TTAAGGACGGCAAAGTTGTAAGTTTTGGCCACTGACTGACTTACAACTGCCGTCCTTAA |
| 36 | TTTAAGGACGGCAAAGTTGTAGTTTTGGCCACTGACTGACTACAACTTCCGTCCTTAAA |
| 37 | ATGTCTTTGGGACTCTGACTCGTTTTGGCCACTGACTGACGAGTCAGACCCAAAGACAT |
| 38 | TATCTATCCTGTGTCTACCAGGTTTTGGCCACTGACTGACCTGGTAGACAGGATAGATA |
| 39 | AAATTACTTTCTGATCCTCAGGTTTTGGCCACTGACTGACCTGAGGATGAAAGTAATTT |
| 40 | TCTAACAAGCCCATGTCTTTGGTTTTGGCCACTGACTGACCAAAGACAGGCTTGTTAGA |
| 41 | AATTACTTTCTGATCCTCAGGGTTTTGGCCACTGACTGACCCTGAGGAAGAAAGTAATT |
| 42 | AGTAAATGTACACAGGCACAGGTTTTGGCCACTGACTGACCTGTGCCTGTACATTTACT |
| 43 | TAAGTAAGGTTGGCTGAGTTAGTTTTGGCCACTGACTGACTAACTCAGAACCTTACTTA |
| 44 | TTCTGTGGGTGAAAGATCCTTGTTTTGGCCACTGACTGACAAGGATCTCACCCACAGAA |
| 45 | AGAAGATGCTGACAACACCCTGTTTTGGCCACTGACTGACAGGGTGTTCAGCATCTTCT |
| 46 | AATTGTAGCCGGCTGGCTAGTGTTTTGGCCACTGACTGACACTAGCCACGGCTACAATT |
| 47 | AGATTTGGGCAAACGCTCTTAGTTTTGGCCACTGACTGACTAAGAGCGTGCCCAAATCT |
| 48 | ATTCTACGCTCCCTTATGCTGGTTTTGGCCACTGACTGACCAGCATAAGAGCGTAGAAT |
| 49 | TGGTAATAGAGAGACCAGAATGTTTTGGCCACTGACTGACATTCTGGTCTCTATTACCA |
| 50 | TATAGATGGCAAGAGGACCAAGTTTTGGCCACTGACTGACTTGGTCCTTGCCATCTATA |
| 51 | AAACCAGTGTAGCTGCAGCCCGTTTTGGCCACTGACTGACGGGCTGCATACACTGGTTT |

The miRNA of the present embodiment may have a nucleotide sequence selected from the group consisting of SEQ ID NOs: 2 to 7 and 24 to 51, preferably has a nucleotide sequence selected from the group consisting of SEQ ID NOs: 2 to 5, 7, 24, 26, 29 to 32, 35, 36, 42, and 51, and more preferably has a nucleotide sequence selected from the group consisting of SEQ ID NOs: 2, 29, 31, and 32.

[Vector]

The oligodendrocyte-specific vector of the present embodiment comprises an oligodendrocyte-specific promoter and/or an miRNA specific to the PLP1 gene. The vector of the present embodiment is a vector for use in treatment of a disease associated with abnormality in the PLP1 gene, or for the purpose of suppression of expression of the PLP1 gene.

The vector needs to be a plasmid or vector capable of infecting human cells and expressing a gene, and examples of the vector include adeno-associated virus (AAV), lentivirus, retrovirus, adenovirus, Sendai virus, and plasmids (including complexes with a liposome or a polymer), and the vector is preferably AAV. AAV is a small, helper-dependent envelope-free virus that is classified into the genus Dependovirus in the family Parvoviridae and infects animals including humans, primates, and rodents. The induction of immunoreaction by AAV is very weak, and no pathogenicity has been found for AAV. AAV is capable of infecting both dividing cells and non-dividing cells, and once infecting host cells, AAV can survive out of the nuclear chromosomes and express a gene with a low frequency of insertion of the gene into the chromosomal genome of the host.

For the AAV, for example, any of the serotypes AAV1, AAV2, AAV3, AAV4, AAVS, AAV6, AAV7, AAV8, and AAV9 is preferably used, and a hybrid mosaic AAV vector and a chimeric AAV vector are also preferably used. For the hybrid mosaic AAV vector, for example, any combinations of AAV1 to AAV9 including an AAV1/2 hybrid are available. Examples of the chimeric AAV vector include Olig001 (Powell S K et al., Gene Ther. 2016 November; 23 (11): 807-814), which has high tropism for oligodendrocytes.

The oligodendrocyte-specific promoter in the vector of the present embodiment is not limited to a particular promoter and may be any promoter that operates specifically in oligodendrocytes, and is preferably the promoter of the present embodiment, that is, the oligodendrocyte-specific promoter is, for example, an oligodendrocyte-specific promoter comprising a nucleic acid preferably having a sequence identity of at least 90% to the nucleotide sequence set forth in SEQ ID NO: 1. The miRNA specific to the PLP1 gene in the vector of the present embodiment is not limited to a particular miRNA and may be any miRNA capable of suppressing expression of the PLP1 gene, and preferably is the miRNA of the present embodiment, that is, the miRNA is an miRNA comprising a pair of nucleotide sequences consisting of an antisense sequence and a sense sequence, wherein the pair of nucleotide sequences is selected from the group consisting of pairs of an antisense sequence set forth in a left column of Table 1 and a sense sequence set forth in the just right of the antisense sequence in a right column of the table.

The vector of the present embodiment may comprise the oligodendrocyte-specific promoter of the present embodiment and a transgene operably linked to the promoter. The transgene is not limited to a particular transgene, and, for example, an miRNA sequence specific to the PLP1 gene, in particular, an miRNA sequence specific to the human PLP1 gene is preferably used. The miRNA sequence specific to the human PLP1 gene can be designed, for example, by using software such as BLOCK-iT™ RNAi Designer (Thermo Fisher Scientific, USA). Alternatively, the miRNA sequence specific to the human PLP1 gene can be designed in such a manner that artificial siRNAs are designed as described in Examples, and an optimum sequence is selected from the siRNAs to design the miRNA. For such an miRNA specific to the human PLP1 gene, for example, the above-described miRNA of the present embodiment is preferably used.

The vector of the present embodiment may comprise an oligodendrocyte-specific promoter and the miRNA of the present embodiment that is operably linked to the promoter. The oligodendrocyte-specific promoter is not limited to a particular promoter, and examples of the oligodendrocyte-specific promoter include a myelin basic protein gene promoter and a PLP1 gene promoter. For the oligodendrocyte-specific promoter, the above-described oligodendrocyte-specific promoter of the present embodiment is preferably used. Preferably, the vector of the present embodiment may comprise the oligodendrocyte-specific promoter of the present embodiment and one or more miRNAs of the present embodiment that are operably linked to the promoter.

The vector of the present embodiment may comprise a reporter gene to confirm gene expression. Examples of the reporter gene include, but are not limited to, GFP, Venus, and tdTomato.

The vector of the present embodiment can be constructed with ease through gene engineering. For example, an AAV1/2 hybrid vector including the hCNP promoter set forth in SEQ ID NO: 1 and a reporter gene can be produced with a method as described in the following. The hCNP promoter, a coding region for a reporter gene such as Venus, a 3′ untranslated region, and an SV40 poly A signal are disposed in the order presented between two NotI sites of a self-complementary AAV vector, and the nucleotide sequence of an miRNA expression cassette is then inserted into the 3′ untranslated region. The vector can be cryopreserved at −20° C. until use, and, when the vector is used, AAV293 cells (Takara Bio Inc., Japan) are transfected with the vector together with a serotype-specific helper plasmid and the adenovirus helper plasmid pHelper, and then cultured for 72 hours to achieve amplification in cells or in the culture solution. Depending on the scale, purification can be performed by means of column purification, ultrafiltration, ultracentrifugation, or the like. The vector genome titer of the AAV vector obtained can be determined through quantitative PCR using an AAVpro Titration Kit (Takara Bio Inc., Japan).

[Pharmaceutical Composition]

The pharmaceutical composition of the present embodiment comprises the above-described vector of the present embodiment. The pharmaceutical composition of the present embodiment is preferably a pharmaceutical composition for treating a disease associated with abnormality in the PLP1 gene. Examples of the disease associated with abnormality involving the PLP1 gene include Pelizaeus-Merzbacher disease (PMD) and spastic paraplegia type 2, each of which is caused by duplication of the PLP1 gene, point mutation in the PLP1 gene, or deletion or insertion mutation involving the PLP1 gene, or multiple sclerosis. In addition, the pharmaceutical composition of the present embodiment may be a PLP1 gene expression suppressor capable of suppressing expression of the PLP1 gene.

The pharmaceutical composition of the present embodiment is preferably an aqueous solution containing an AAV vector, and may have a titer of 5×10¹⁰ to 5×10¹⁴ vg/mL, preferably of 5×10¹¹ to 5×10¹³ vg/mL.

The pharmaceutical composition of the present embodiment may contain a pharmaceutically acceptable additive component such as phosphate-buffered saline (PBS), and may further contain an additional therapeutic agent. The pharmaceutical composition of the present embodiment can be produced by dissolving in an aqueous solution such as water and adding other components, as necessary.

The pharmaceutical composition of the present embodiment may be directly administered to the brain in treating Pelizaeus-Merzbacher disease, and examples of such administration include administration to one site or multiple sites of the brain parenchyma (e.g., the white matter in the cerebral hemisphere, the internal capsule, the cerebellum) or the spinal cord, and intracerebroventricular/intrathecal or intravascular/intraperitoneal administration is also acceptable. Administration can be performed at any time after birth. The amount of injection can vary among administration sites and ages, and an amount of 0.1 to 2 mL/site is appropriate in the case of administration to the brain parenchyma, and it is preferred to administer about 0.5 mL/site. In the case of intracerebroventricular/intrathecal administration, an amount of 0.5 to 2 mL/kg body weight is appropriate, and it is preferred to administer up to about 1 mL/kg body weight. In the case of intravascular or intraperitoneal administration, an amount of 1 to 10 mL/kg body weight is appropriate, and it is preferred to administer up to 5 mL/kg body weight.

[Therapeutic Method]

The therapeutic method of the present embodiment is a method for treating a disease associated with abnormality of the PLP1 gene, the method comprising administering an effective amount of the above-described vector of the present embodiment to a patient in need of treatment. Alternatively, the therapeutic method of the present embodiment is a method for suppressing expression of the PLP1 gene, the method comprising administering an effective amount of the above-described vector of the present embodiment to a patient in need of treatment.

### EXAMPLES

Hereinafter, the present invention will be more specifically described with reference to Examples. However, the present invention is not limited to Examples in the following.

### Example 1 Construction of Oligodendrocyte-Specific AAV Expression Cassette and AAV Vector Including the Same

A pre-miRNA sequence (PLP1 miRNA, SEQ ID NO: 9) targeting the mRNA of a mouse PLP1 gene (NM 011123.3, SEQ ID NO: 8) and a pre-miRNA sequence as a negative control (miR-neg, SEQ ID NO: 10) were designed by using BLOCK-iT™ RNAi Designer (Thermo Fisher Scientific, USA). The miR-neg is an miRNA sequence that can form a hairpin structure, thereby processed into a mature miRNA, but is predicted not to target known vertebrate genes.

PLP1-miRNA (SEQ ID NO: 9):
5′-
ACTCCAAAGAAACACAATCCAGTTTTGGCCACTGACTGACTG
GATTGTTTCTTTGGAGT-3′
miR-neg (SEQ ID NO: 10):
5′-
GTATGCATCGAATGAGATTCCGTTTTGGCCACTGACTGACGGA
ATCTCTCGATGCATAC-3′

Each of The PLP1 miRNA sequence and the miR-neg sequence was synthesized as designed, and cloned into a cloning site of a pcDNA™ 6.2-GW/miR vector (Thermo Fisher Scientific, USA) included in a BLOCK-iT™ Pol II miR RNAi expression vector kit. A 5′ flanking sequence (SEQ ID NO: 11) and 3′ flanking sequence (SEQ ID NO: 12) derived from mouse miR-155 were respectively disposed at one side and the other side of the PLP1 miRNA sequence or miR-neg sequence. The 5′ flanking sequence and 3′ flanking sequence derived from mouse miR-155 are respectively the 5′ region and 3′ region of mouse miR-155 with a portion of hairpin structure excluded therefrom, and the pre-miRNA sequence or miR-neg sequence was disposed between the 5′ flanking sequence and the 3′ flanking sequence so that these miRNA sequences could be expressed.

5′ flanking sequence (SEQ ID NO: 11):
5′-CTGGAGGCTTGCTGAAGGCTGTATGCT-3′
3′ flanking sequence (SEQ ID NO: 12):
5′-
CAGGACACAAGGCCTGTTACTAGCACTCACATGGAACAAATG
GCC-3′

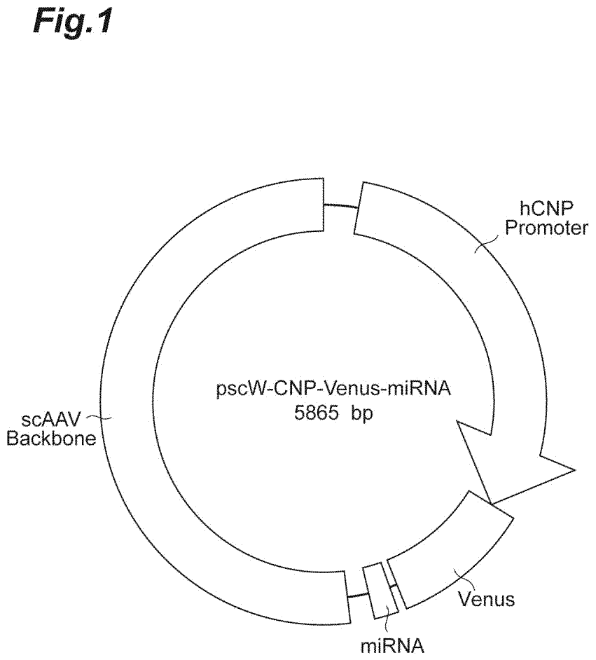

From the resulting pcDNA™ 6.2-GW/miR vector, the portion of 5′ flanking sequence-PLP1 miRNA sequence (or miR-neg sequence)-3′ flanking sequence was PCR-amplified, the amplified product was cleaved with XhoI/BamHI, and the resultant was inserted into a DNA fragment obtained by cleaving a pW-CAG-Venus-WPRE vector (SEQ ID NO: 13) with XhoI/BamHI and cleaved at both ends of WPRE positioned in the 3′ side of a venus (SEQ ID NO: 14) sequence to remove the WPRE sequence. Subsequently, the resulting vector was cleaved with SpeI/EcoRI to remove the CAG promoter, and a DNA fragment was inserted into this site in the 5′ side of the Venus sequence, the DNA fragment being obtained by PCR-amplifying a human CNP promoter (SEQ ID NO: 1) of 1.8 kb and cleaved at the both ends with SpeI/EcoRI, to enable oligodendrocyte-specific gene expression. The resulting vector was further cleaved with NotI to remove the backbone of single-stranded AAV derived from pW-CAG-Venus-WPRE, and bonded to the backbone of self-complementary adeno-associated virus (scAAV) obtained by cleaving pscW-PABPN1 (SEQ ID NO: 15) with NotI to afford the final constructs Pscw-hCNP-Venus-PLP1 miRNA (SEQ ID NO: 16) and Pscw-hCNP-Venus-miR-neg (SEQ ID NO: 17) (FIG. 1).

Preparation of an AAV1/2 hybrid vector (scAAV-AAV1/2 vector) was performed in accordance with a method disclosed in Non Patent Literature 1. Specifically, AAV293 cells were co-transfected using PEI with the Pscw-hCNP-Venus-PLP1 miRNA or Pscw-hCNP-Venus-miR-neg, which was an scAAV construct obtained above, the serotype-specific AAV helper plasmids p5E18RXC 1 (Xiao W et al., J Virol (1999) 73: 3994-4003) and pAAV-RC (Agilent Technologies) encoding the rep and cap genes of AAV1 and AAV2, respectively, and the adenovirus helper plasmid pHelper (Agilent Technologies). The cells were collected 72 hours after the transfection, and each scAAV-AAV1/2 vector was purified by using an AAVpro Purification Kit (Takara Bio Inc., Japan). Vector genome titers were determined through quantitative PCR using an AAVpro Titration Kit (Takara Bio Inc., Japan).

The scAAV-AAV1/2 vectors obtained were designated as the AAV-hCNP-Venus-PLP1 miRNA vector (or PLP1 miRNA vector) and the AAV-hCNP-Venus-miR-neg vector (or miR-neg vector).

### <Example 2 Administration of AAV Vectors to Wild-Type Mice and Expression Thereof

One microliter of a solution of the PLP1 miRNA vector or miR-neg vector obtained in Example 1 (titer: 1.2×10¹² vg/mL) was injected into one striatum and the internal capsule of each 10-day-old, wild-type mouse Jc1:B6C3F1 (n=5 per group). Before the injection, the mice were anesthetized with pentobarbital solution. The injection of the vector was performed with a 33G syringe at a rate of 150 nL/min After the injection each mouse was left untouched there for 1 minute, and the needle was slowly pulled out of the brain. Thereafter, the mice were returned as a group to their mother, and grown until day 17 after birth.

Gene expression from each vector was evaluated by detecting the green fluorescent protein Venus on day 17 after birth. Types of Venus-expressing cells were identified through double immunostaining with cell markers including Gst-π (for mature oligodendrocytes), NeuN (for neurons), IbaI (for microglia), and GFAP (astrocytes).

For immunostaining, each Jc1:B6C3F1 mouse was anesthetized with Forane inhalation solution, and subsequently subjected to transcardial perfusion with PBS and then with fresh 4% paraformaldehyde. The brain was removed and further fixed at 4° C. overnight, and then subjected to substitution with 30% sucrose/PBS and cryopreserved in the embedding medium OCT Compound (TissueTech, Inc.). A coronal section (20 μm) was obtained, and immunostained to check specificity of infected cells expressing Venus. The section was incubated together with blocking solution (PBS containing 0.05% Triton™ X-100 and 5% goat serum) at room temperature for 3 hours, and incubated at 4° C. overnight with Gst-π (Cell Signaling Technology, Inc., 1:300), GFAP (Millipore Corporation, 1:400), NeuN (Chemicon, 1:400), or IbaI (Biocare Medical, LLC., 1:500), as primary antibodies. The section was then washed, and incubated together with an appropriate secondary antibody (Thermo Fisher Scientific, USA) at room temperature for 1 hour. Cell nuclei were visualized with 40,60-diamidino-2-phenylindole (DAPI, Sigma-Aldrich Co. LLC). The slide was mounted with ProLong (R) Diamond Mountant (Thermo Fisher Scientific, USA), and photographed with a KEYENCE fluorescence microscope (KEYENCE CORPORATION, Japan).

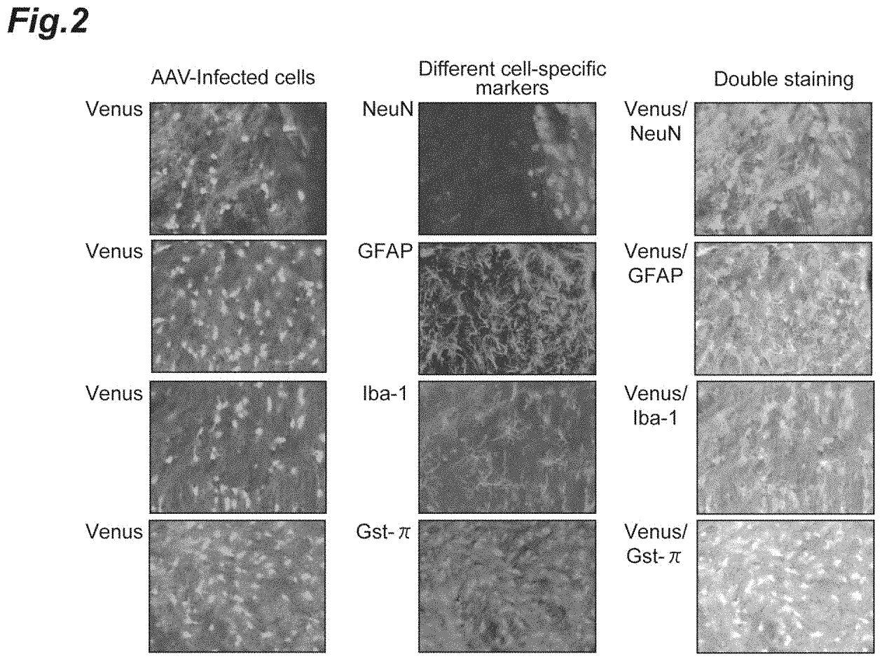

FIG. 2 shows the results. It is understood from FIG. 2 that Venus-positive cells corresponded to Gst-π-positive oligodendrocytes but did not correspond to NeuN-positive cell neurons, GFAP-positive astrocytes, or IbaI-positive microglia. Thus, FIG. 2 demonstrates that the miR-neg vector was expressed in an oligodendrocyte-specific and highly efficient manner.

### Example 3 Evaluation of Suppression Efficiency of Expression (Knockdown) in Terms of PLP1 Gene Expression Levels—Part 1

One microliter of a solution of each of the two scAAV-AAV1/2 vectors (titer: 1.2×10¹² vg/mL) obtained in Example 1 was injected into one striatum, the internal capsule, and the cerebellum of each 10-day-old, wild-type mouse Jc1:B6C3F1 (n=3 per group). The injection method was the same as in Example 2. Thereafter, expression of PLP1 protein in Venus-positive cells was examined through immunostaining to evaluate PLP1 miRNA knockdown efficiencies. Immunostaining was performed with the same method as in Example 2 except that an anti-PLP1 rabbit polyclonal antibody (Numata Y et al., J Biol Chem. 2013 Mar. 15; 288 (11): 7451-66) was used as a primary antibody, and an anti-rabbit fluorescent antibody (Thermo Fisher Scientific, USA) was used as a secondary antibody. Each site of the callosum, striatum, and internal capsule were photographed with a KEYENCE fluorescence microscope (KEYENCE CORPORATION, Japan).

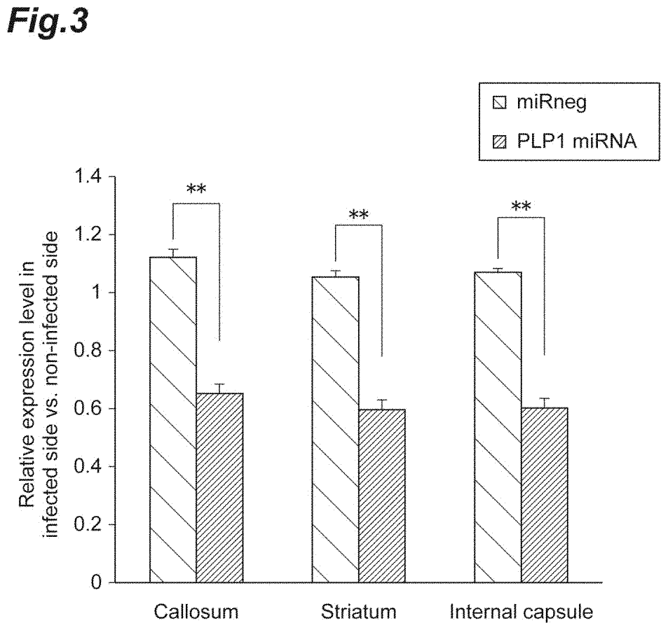

Mean fluorescence intensities of immunostained PLP1 were quantified by using Image J 1.45s (Wayne Rasband, National Institutes of Health, USA), and FIG. 3 shows quantitative analysis of fluorescence intensity through PLP1 immunofluorescence staining. It can be seen from FIG. 3 that there was almost no difference in expression of PLP1 protein between the side without injection of the miR-neg vector (non-infected side) and the side with vector injection (infected side), whereas for the PLP1 miRNA vector there was 40% of expression-suppressing effect on PLP1 protein in the infected side as compared with the non-infected side. The result that there was no difference in the number of astrocytes and that of microglia between the non-infected side and the infected side indicates the absence of reactive inflammatory response due to AAV administration.

### Example 4 Evaluation of Suppression Efficiency of Expression (Knockdown) in Terms of PLP1 Gene Expression Levels—Part 2

One microliter of a solution of each of the two scAAV-AAV1/2 vectors (titer: 1.2×10¹² vg/mL) obtained in Example 1 was injected into both striata and the internal capsule of each 10-day-old, wild-type mouse Jc1:B6C3F1 (n=4 per group). The injection method was the same as in Example 2. Thereafter, expression of the PLP1 mRNA in Venus-positive cells was examined through quantitative PCR to evaluate PLP1 miRNA knockdown efficiency.

To sort Venus-positive cells, each Jc1:B6C3F1 mouse was anesthetized with Forane inhalation solution, and subjected to transcardial perfusion with 20 mL of PBS. The brain was removed, and roughly fragmented with micro dice scissors. To each brain, 1 mL of Accutase (Millipore Corporation) was added, and the resultant was incubated at 37° C. for 30 minutes. To each sample, 2 mL of Hanks' balanced salt solution (HBSS) containing 10% FBS was added, and the tissue was homogenized with a micropipette. Each sample was filtered with a 100-μm cell strainer, and the collected cell suspension was centrifuged. To purify cells from myelin debris, cells were resuspended in HBSS containing 40% Percoll (Amersham plc), and centrifuged at 700×g at room temperature for 25 minutes. The upper myelin layer was sucked, and monocytes were resuspended in 1 mL of a DMEM/F12 medium. These cells were separated by an FACS Canto flow cytometer (BD Biosciences), and the result was analyzed by using the software FlowJo (Tree Star Inc., OR, RRID: NIF-0000 to 30575). The result is plotted as percent to all cells.

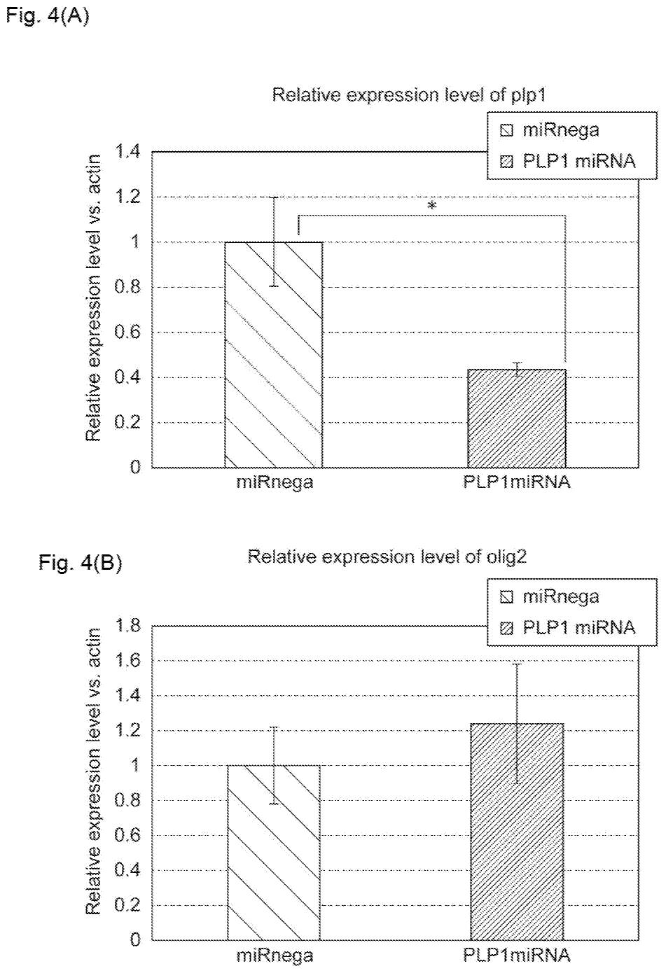

For quantitative PCR, the total RNA was isolated from the selected Venus-positive cells using an RNeasy Mini Kit (Qiagen), and a cDNA was synthesized by using a SuperScript (R) Reverse Transcriptase Kit (Thermo Fisher Scientific, USA). Gene expression was analyzed through quantitative RT-PCR using LightCycler (R) 480 SYBR Green I Master and a LightCycler (R) 480 Instrument with specific primers below (F. Hoffmann-La Roche Ltd., Switzerland). The results were normalized to the house keeping gene β-actin, and relative expression levels of PLP1 and Olig2 were calculated. CP values (crossing point values) obtained for each specimen were converted into log values to plot relative expression levels±SD, and the plotted results are shown in FIG. 4.

PLP1 forward primer (SEQ ID NO: 18):
5′-GTTCCAGAGGCCAACATCAAGCTC-3′
PLP1 reverse primer (SEQ ID NO: 19):
5′-AGCCATACAACAGTCAGGGCATAG-3′
Olig2 forward primer (SEQ ID NO: 20):
5′-GGGAGGTCATGCCTTACGC-3′
Olig2 reverse primer (SEQ ID NO: 21):
5′-CTCCAGCGAGTTGGTGAGC-3′
β-actin forward primer (SEQ ID NO: 22):
5′-CACAGCTTCTTTGCAGCTCCTT-3′
β-actin reverse primer (SEQ ID NO: 23):
5′-GACGACCAGCGCAGCGATA-3′

It can be seen from FIG. 4 that for the expression level of the mRNA of Olig2, an oligodendrocyte marker, there was no significant difference between the miR-neg vector and the PLP1 miRNA vector (B), whereas for the expression level of the PLP1 mRNA, injection of the PLP1 miRNA vector provided approximately 60% of expression-suppressing effect on the PLP1 gene (A).

### Example 5 Effect of Administration to PLP1 Transgenic (PLP1-Tg) Mice

To both striata and the internal capsule of each of a litter of 10-day-old (body weight: 5 to 6 g) PLP1-Tg/B6C3 mice overexpressing mouse PLP1, the PLP1 miRNA vector (group with treatment, n=16) or the miR-neg vector (group with mock treatment, n=16) was injected to conduct treatment test. PLP1-Tg/B6C3 was prepared by backcrossing PLP1-Tg/BDF1, which was gifted from Dr. Tetsushi Kagawa in National Institute for Physiological Sciences, with a B6C3 strain (Kagawa T et al., Neuron. 1994 August; 13 (2): 427-42). For PLP1-Tg-homozygous individuals, 1 μL of a solution of each of the two scAAV-AAV1/2 vectors (titer: 1.2×10¹² vg/mL) obtained in Example 1 was injected into both striata and the internal capsule of each 10-day-old mouse in the same manner as in Example 2. For a positive control, wild-type litter mate mice injected with the miR-neg vector (n=16) were used. Some of those mice were removed of brain tissue on day 25 after birth (15 days after the injection) and histological analysis (immunostaining, morphometric analysis) was conducted for the brain tissue, and the residual mice were observed for body weights and survival rates.

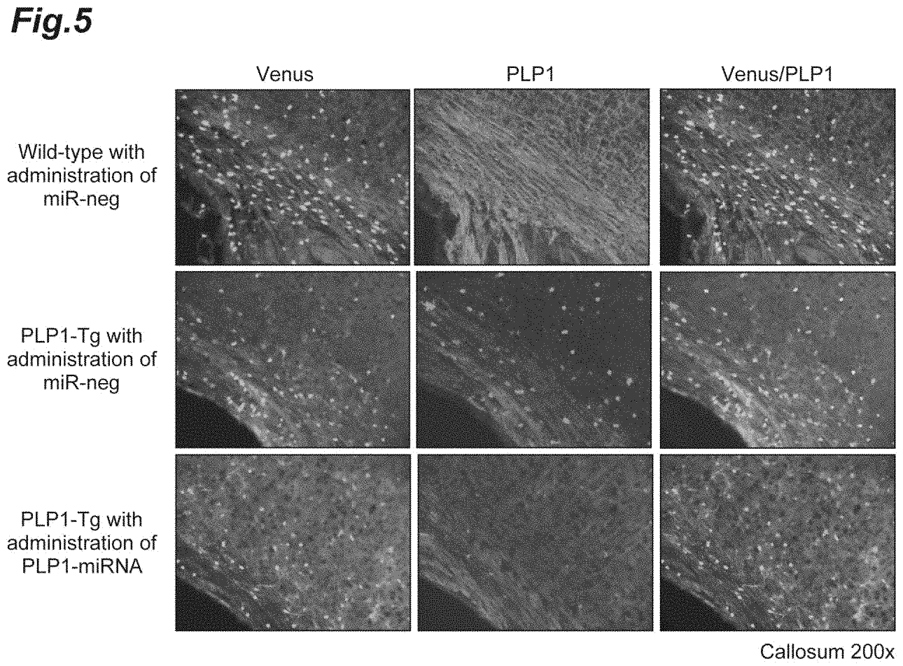

Histological analysis by immunostaining was carried out as follows. For the group of wild-type mice, and the group of PLP1-Tg mice with treatment and that with mock treatment, where n=5 per group, each of the callosum, striatum, and internal capsule were immunostained in the same manner as in Example 3 on day 25 after birth (15 days after the injection). For the wild-type mice as a positive control group, PLP1 provided fibrous, strongly stained images corresponding to myelin sheaths in the callosum, striatum, and internal capsule. In analysis of the PLP1-Tg mice, almost no stained image corresponding to myelin sheaths was found for the mice with mock treatment, whereas a stained image indicating abnormal accumulation of PLP1 in the cytoplasm of oligodendrocytes were obtained. For the mice with treatment, by contrast, almost no stained image indicating the accumulation of PLP1 in cytoplasm was found, and a fibrous stained image inferred to correspond to myelin sheaths were found, which revealed that administration of the PLP1 miRNA vector provides improved stained images of PLP1 as compared with abnormal ones. FIG. 5 shows micrographs of callosum.

Micromorphological analysis with an electron microscope was performed in the following manner. For the wild-type mice and the group of PLP1-Tg mice with treatment and that with mock treatment, where n=2 per group, fixing by reflux was performed with 0.1 M phosphate buffer containing 2% glutaraldehyde and 2% paraformaldehyde on day 25 after birth (15 days after the injection), and then the brain was removed and further fixed by soaking in the same buffer. Each of the callosum, striatum, and internal capsule was checked for AAV-infected sites under a stereoscopic fluorescence microscope, a tissue section of 600-μm square was cut out, and this was post-fixed with 0.1 M phosphate buffer containing 1% osmium. After dehydration, the tissue section was embedded in epoxy resin, a 70-nm ultrathin section was prepared, and this was observed through an electron microscope (Tecnai Spirit, Thermo Fisher Scientific, USA) at a magnification of 7300×.

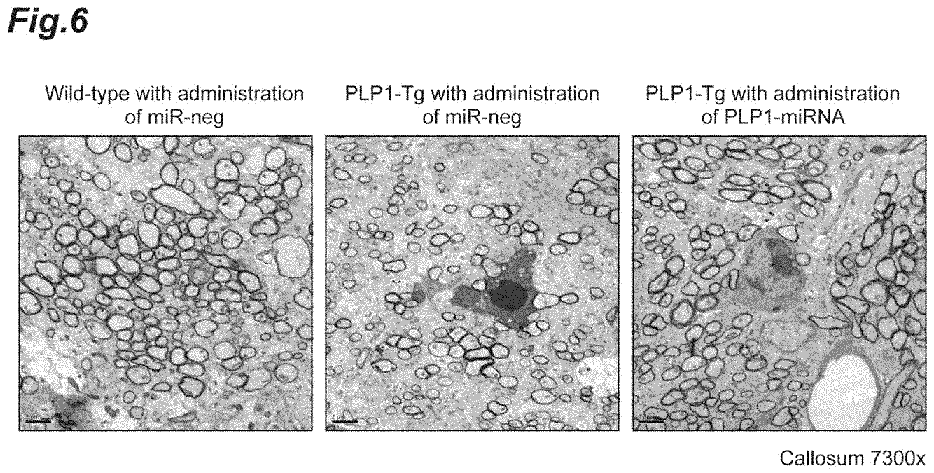

FIG. 6 shows electron micrographs. It was clear that for the wild-type mice almost all axons were myelinated in each of the callosum, striatum, and internal capsule. In analysis of the PLP1-Tg mice, the fraction of myelinated axons was clearly low in the mice with mock treatment, and moreover the axons were narrowed. In the mice with treatment, on the other hand, the fraction of myelinated axons was higher, and moreover diameters of axons were generally larger than those in the mice with mock treatment. From these results, it was revealed from the viewpoint of ultrafine morphology that administration of the PLP1 miRNA vector provides therapeutic effect owing to enrichment of myelinated fibers.

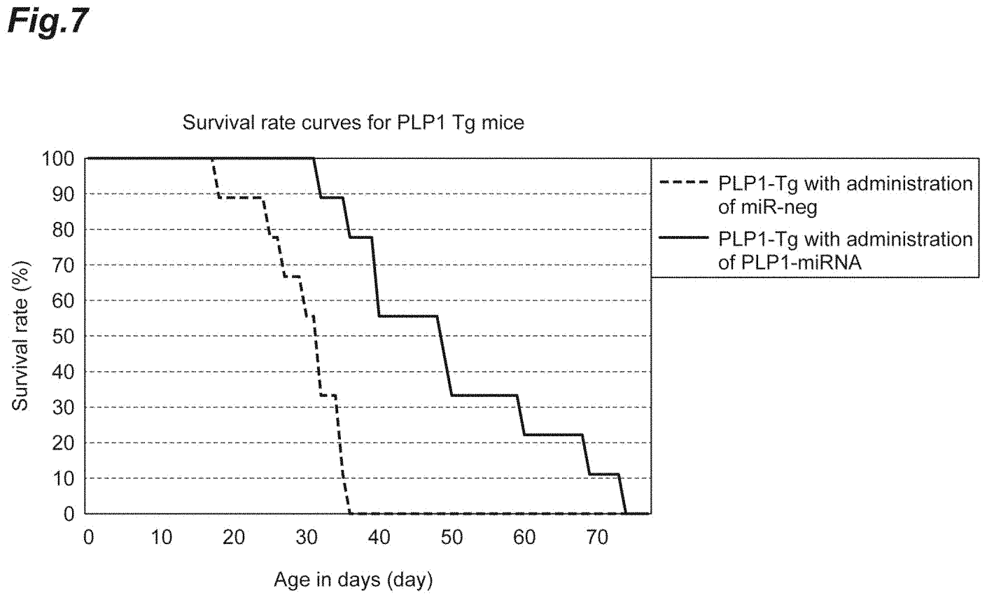

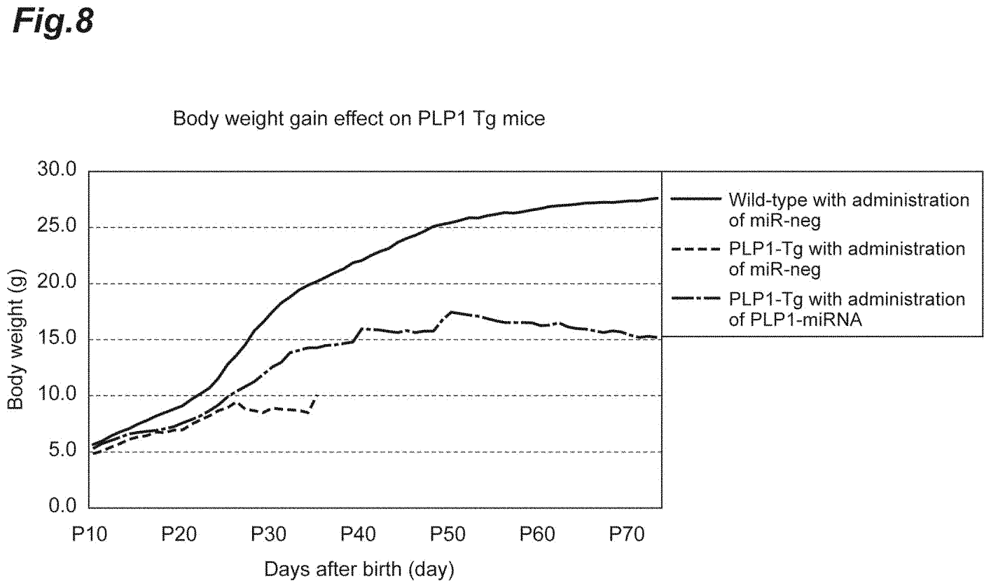

The group of mice with treatment and group of mice with mock treatment, where n=9 per group, were observed even after 25 days in age until their death to evaluate body weights and survival rates for the groups. Administration to the PLP1-Tg mice was found to provide significant survival rate-improving (life-prolonging) effect (FIG. 7) and body weight gain effect (FIG. 8), and thus the efficacy of the present therapy was confirmed.

The thus-described results revealed that the oligodendrocyte-specific promoter and the AAV vector including the promoter can be applicable as a fundamental technique to develop therapy for PMD, and that the gene expression AAV vector with a PLP1-specific miRNA using the promoter can be an effective approach for treatment of PMD.

### Example 6 Evaluation of Suppression Efficiency of Expression (Knockdown) in Terms of PLP1 Gene Expression Levels—Part 3

To evaluate the knockdown efficiency of the human PLP1-miRNA designed with BLOCK-iT™ RNAi Designer (Thermo Fisher Scientific, USA), pcDNA™ 6.2-hPLP1, which is a plasmid capable of overexpressing a sequence encoding the human PLP1 gene, and pscw.CAG.Venus.PLP1-miRNA, which is a plasmid obtained by cloning a sequence into a 3′ untranslated region in the downstream of a cDNA encoding Venus, a fluorescent protein that is expressed under the control of a CAG promoter, were concomitantly introduced into HeLa cells through a transfection method. For the transfection, TransIt-LT1 (Takara Bio Inc.) was used. After 24 hours cells were collected, and the total RNA was extracted by using an RNeasy Mini Kit (Qiagen). For quantitative PCR, a cDNA was synthesized by using a SuperScript (R) Reverse Transcriptase Kit (Thermo Fisher Scientific, USA). Gene expression was analyzed through quantitative PCR using LightCycler (R) 480 SYBR Green I Master and a LightCycler (R) 480 Instrument with specific primers below (F. Hoffmann-La Roche Ltd., Switzerland). The primers used in the quantitative PCR for PLP1 gene expression are listed in the following.

Human PLP1 forward primer (SEQ ID NO: 280):
5′-GCTCCAACCTTCTGTCCATCT-3′
Human PLP1 reverse primer (SEQ ID NO: 281):
5′-ACGGCAAAGTTGTAAGTGGC-3′
Human 13-actin forward primer (SEQ ID NO: 282):
5′-GACAGGATGCAGAAGGAGATTACT-3′
Human 13-actin reverse primer (SEQ ID NO: 283):
5′-TGATCCACATCTGCTGGAAGGT-3′

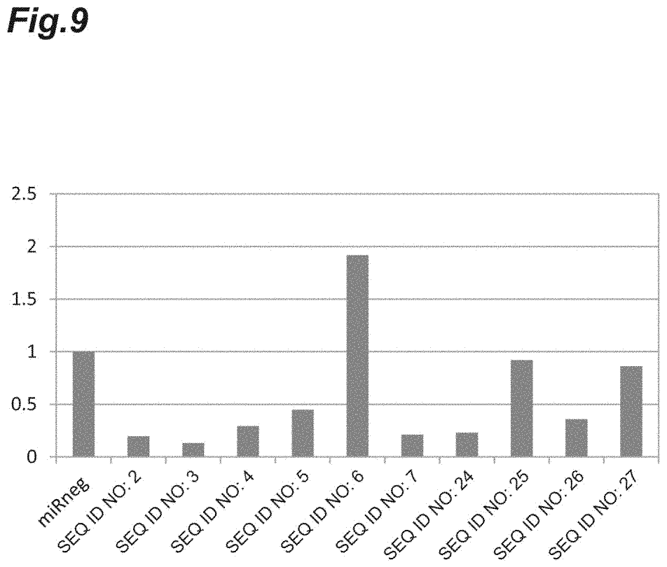

FIG. 9 shows results of the knockdown. Knockdown efficiencies were calculated assuming the PLP1 expression level of cells with miR-neg expression as 1. The results were normalized to the house keeping gene β-actin, and calculated as relative expression levels±SD. Seven sequences that lower PLP1 gene expression levels were identified (SEQ ID NOs: 2, 3, 4, 5, 7, 24, and 26).

### Example 7 Evaluation of Suppression Efficiency of Expression (Knockdown) in Terms of PLP1 Gene Expression Levels—Part 4

A pair of siRNAs consisting of a sense strand and the corresponding antisense strand shown in Table 3 and transfection solution (RNAi MAX, Invitrogen) were mixed together, and the solution prepared was added to U-251MG cells, and the cells were cultured for 48 hours.

TABLE 3
siRNA Sequences designed for human PLP1 gene

| | SEQ | | |
|---|---|---|---|
| | ID | Sense/ | |
| No. | NO: | Antisense strand | siRNA Sequence |
| 1 | 118 | hPLP1-001 | UGACCUUCCACCUGUUUAUdTdT |
| | 119 | hPLP1-001anti | AUAAACAGGUGGAAGGUCAdTdT |
| 2 | 120 | hPLP1-002 | GUGCCUGUGUACAUUUACUdTdT |
| | 121 | hPLP1-002anti | AGUAAAUGUACACAGGCACdTdT |
| 3 | 122 | hPLP1-003 | UUGCCCAAAUCUGCCUAUUdTdT |
| | 123 | hPLP1-003anti | AAUAGGCAGAUUUGGGCAAdTdT |
| 4 | 124 | hPLP1-004 | AGGAAUGGAGGCUCUAAUUdTdT |
| | 125 | hPLP1-004anti | AAUUAGAGCCUCCAUUCCUdTdT |
| 5 | 126 | hPLP1-005 | UGGCCACUGGAUUGUGUUUdTdT |
| | 127 | hPLP1-005anti | AAACACAAUCCAGUGGCCAdTdT |
| 6 | 128 | hPLP1-006 | GCGGGUGUGUCAUUGUUUGdTdT |
| | 129 | hPLP1-006anti | CAAACAAUGACACACCCGCdTdT |
| 7 | 130 | hPLP1-007 | GACCUUCCACCUGUUUAUUdTdT |
| | 131 | hPLP1-007anti | AAUAAACAGGUGGAAGGUCdTdT |
| 8 | 132 | hPLP1-008 | ACUCCCUUCUCCUUGAUAAdTdT |
| | 133 | hPLP1-008anti | UUAUCAAGGAGAAGGGAGUdTdT |
| 9 | 134 | hPLP1-009 | CUGCAAGUCACAAAGGAAUdTdT |
| | 135 | hPLP1-009anti | AUUCCUUUGUGACUUGCAGdTdT |
| 10 | 136 | hPLP1-010 | GGUCCUCUUGCCAUCUAUAdTdT |
| | 137 | hPLP1-010anti | UAUAGAUGGCAAGAGGACCdTdT |
| 11 | 138 | hPLP1-011 | CCUCGUUAGGGAAGAGAAAdTdT |
| | 139 | hPLP1-011anti | UUUCUCUUCCCUAACGAGGdTdT |
| 12 | 140 | hPLP1-012 | GGAGAAGAGGACAAAGAUAdTdT |
| | 141 | hPLP1-012anti | UAUCUUUGUCCUCUUCUCCdTdT |
| 13 | 142 | hPLP1-013 | GGCCAACAUCAAGCUCAUUdTdT |
| | 143 | hPLP1-013anti | AAUGAGCUUGAUGUUGGCCdTdT |
| 14 | 144 | hPLP1-014 | AGGCCAACAUCAAGCUCAUdTdT |
| | 145 | hPLP1-014anti | AUGAGCUUGAUGUUGGCCUdTdT |
| 15 | 146 | hPLP1-015 | CUCCCUUCUCCUUGAUAACdTdT |
| | 147 | hPLP1-015anti | GUUAUCAAGGAGAAGGGAGdTdT |
| 16 | 148 | hPLP1-016 | GCCUCUUUCUUCUUCCUUUdTdT |
| | 149 | hPLP1-016anti | AAAGGAAGAAGAAAGAGGCdTdT |
| 17 | 150 | hPLP1-017 | CUGUGCCUGUGUACAUUUAdTdT |
| | 151 | hPLP1-017anti | UAAAUGUACACAGGCACAGdTdT |
| 18 | 152 | hPLP1-018 | AAGGAAUGGAGGCUCUAAUdTdT |
| | 153 | hPLP1-018anti | AUUAGAGCCUCCAUUCCUUdTdT |
| 19 | 154 | hPLP1-019 | AGGGUGUUGUCAGCAUCUUdTdT |
| | 155 | hPLP1-019anti | AAGAUGCUGACAACACCCUdTdT |
| 20 | 156 | hPLP1-020 | ACAGCUGAGUUCCAAAUGAdTdT |
| | 157 | hPLP1-020anti | UCAUUUGGAACUCAGCUGUdTdT |
| 21 | 158 | hPLP1-021 | GCCACUGGAUUGUGUUUCUdTdT |
| | 159 | hPLP1-021anti | AGAAACACAAUCCAGUGGCdTdT |
| 22 | 160 | hPLP1-022 | ACAACUUUGCCGUCCUUAAdTdT |
| | 161 | hPLP1-022anti | UUAAGGACGGCAAAGUUGUdTdT |
| 23 | 162 | hPLP1-023 | GCUGAUGCCAGAAUGUAUGdTdT |
| | 163 | hPLP1-023anti | CAUACAUUCUGGCAUCAGCdTdT |
| 24 | 164 | hPLP1-024 | UUGCCGUCCUUAAACUCAUdTdT |
| | 165 | hPLP1-024anti | AUGAGUUUAAGGACGGCAAdTdT |
| 25 | 166 | hPLP1-025 | CUGCCUCUUUCUUCUUCCUdTdT |
| | 167 | hPLP1-025anti | AGGAAGAAGAAAGAGGCAGdTdT |
| 26 | 168 | hPLP1-026 | CCACUGGAUUGUGUUUCUUdTdT |
| | 169 | hPLP1-026anti | AAGAAACACAAUCCAGUGGdTdT |
| 27 | 170 | hPLP1-027 | GGAUCAGAAAGUAAUUUCUdTdT |
| | 171 | hPLP1-027anti | AGAAAUUACUUUCUGAUCCdTdT |
| 28 | 172 | hPLP1-028 | UAGCCAGCCGGCUACAAUUdTdT |
| | 173 | hPLP1-028anti | AAUUGUAGCCGGCUGGCUAdTdT |
| 29 | 174 | hPLP1-029 | AGCGUAGAAUCUGUGUAGAdTdT |
| | 175 | hPLP1-029anti | UCUACACAGAUUCUACGCUdTdT |
| 30 | 176 | hPLP1-030 | UCCCUUCUCCUUGAUAACAdTdT |
| | 177 | hPLP1-030anti | UGUUAUCAAGGAGAAGGGAdTdT |
| 31 | 178 | hPLP1-031 | UUCCAAGGAGCUGAGAAUAdTdT |
| | 179 | hPLP1-031anti | UAUUCUCAGCUCCUUGGAAdTdT |
| 32 | 180 | hPLP1-032 | UGCCAGAAUGUAUGGUGUUdTdT |
| | 181 | hPLP1-032anti | AACACCAUACAUUCUGGCAdTdT |
| 33 | 182 | hPLP1-033 | AUGCCUUCCAGUAUGUCAUdTdT |
| | 183 | hPLP1-033anti | AUGACAUACUGGAAGGCAUdTdT |
| 34 | 184 | hPLP1-034 | CAACUUUGCCGUCCUUAAAdTdT |
| | 185 | hPLP1-034anti | UUUAAGGACGGCAAAGUUGdTdT |
| 35 | 186 | hPLP1-035 | CCAACAUCAAGCUCAUUCUdTdT |
| | 187 | hPLP1-035anti | AGAAUGAGCUUGAUGUUGGdTdT |
| 36 | 188 | hPLP1-036 | UGGCCUUACACCUCGUUAGdTdT |
| | 189 | hPLP1-036anti | CUAACGAGGUGUAAGGCCAdTdT |
| 37 | 190 | hPLP1-037 | AGGACAUCCCGACAAGUUUdTdT |
| | 191 | hPLP1-037anti | AAACUUGUCGGGAUGUCCUdTdT |
| 38 | 192 | hPLP1-038 | GUCAGAGUCCCAAAGACAUdTdT |
| | 193 | hPLP1-038anti | AUGUCUUUGGGACUCUGACdTdT |
| 39 | 194 | hPLP1-039 | UUGCAGGAGAAGAGGACAAdTdT |
| | 195 | hPLP1-039anti | UUGUCCUCUUCUCCUGCAAdTdT |
| 40 | 196 | hPLP1-040 | GGUAGACACAGGAUAGAUAdTdT |
| | 197 | hPLP1-040anti | UAUCUAUCCUGUGUCUACCdTdT |
| 41 | 198 | hPLP1-041 | UCAGCCAACCUUACUUACAdTdT |
| | 199 | hPLP1-041anti | UGUAAGUAAGGUUGGCUGAdTdT |
| 42 | 200 | hPLP1-042 | AUGGAACUGCCUCUUUCUUdTdT |
| | 201 | hPLP1-042anti | AAGAAAGAGGCAGUUCCAUdTdT |
| 43 | 202 | hPLP1-043 | ACUCAGCCAACCUUACUUAdTdT |
| | 203 | hPLP1-043anti | UAAGUAAGGUUGGCUGAGUdTdT |
| 44 | 204 | hPLP1-044 | CUUCCACUGAUGGAAACAAdTdT |
| | 205 | hPLP1-044anti | UUGUUUCCAUCAGUGGAAGdTdT |
| 45 | 206 | hPLP1-045 | AUGACCUUCCACCUGUUUAdTdT |
| | 207 | hPLP1-045anti | UAAACAGGUGGAAGGUCAUdTdT |
| 46 | 208 | hPLP1-046 | UCUCCUGAGGAUCAGAAAGdTdT |
| | 209 | hPLP1-046anti | CUUUCUGAUCCUCAGGAGAdTdT |
| 47 | 210 | hPLP1-047 | GAGGAUCAGAAAGUAAUUUdTdT |
| | 211 | hPLP1-047anti | AAAUUACUUUCUGAUCCUCdTdT |
| 48 | 212 | hPLP1-048 | GGAUCUUUCACCCACAGAAdTdT |
| | 213 | hPLP1-048anti | UUCUGUGGGUGAAAGAUCCdTdT |
| 49 | 214 | hPLP1-049 | CUGAGGAUCAGAAAGUAAUdTdT |
| | 215 | hPLP1-049anti | AUUACUUUCUGAUCCUCAGdTdT |
| 50 | 216 | hPLP1-050 | CUGGUAGACACAGGAUAGAdTdT |
| | 217 | hPLP1-050anti | UCUAUCCUGUGUCUACCAGdTdT |
| 51 | 218 | hPLP1-051 | GCAGUUGCUGGUGGCUAAUdTdT |
| | 219 | hPLP1-051anti | AUUAGCCACCAGCAACUGCdTdT |
| 52 | 220 | hPLP1-052 | AAGACAUGGGCUUGUUAGAdTdT |
| | 221 | hPLP1-052anti | UCUAACAAGCCCAUGUCUUdTdT |
| 53 | 222 | hPLP1-053 | UUGCUGCCACUUACAACUUdTdT |
| | 223 | hPLP1-053anti | AAGUUGUAAGUGGCAGCAAdTdT |
| 54 | 224 | hPLP1-054 | UCGUUAGGGAAGAGAAACAdTdT |
| | 225 | hPLP1-054anti | UGUUUCUCUUCCCUAACGAdTdT |
| 55 | 226 | hPLP1-055 | UCCACUGAUGGAAACAAAGdTdT |
| | 227 | hPLP1-055anti | CUUUGUUUCCAUCAGUGGAdTdT |
| 56 | 228 | hPLP1-056 | AGAGCGUUUGCCCAAAUCUdTdT |
| | 229 | hPLP1-056anti | AGAUUUGGGCAAACGCUCUdTdT |
| 57 | 230 | hPLP1-057 | UGAUUGCUGCCACUUACAAdTdT |
| | 231 | hPLP1-057anti | UUGUAAGUGGCAGCAAUCAdTdT |
| 58 | 232 | hPLP1-058 | GGAGCGUAGAAUCUGUGUAdTdT |
| | 233 | hPLP1-058anti | UACACAGAUUCUACGCUCCdTdT |
| 59 | 234 | hPLP1-059 | UGAGGAUCAGAAAGUAAUUdTdT |
| | 235 | hPLP1-059anti | AAUUACUUUCUGAUCCUCAdTdT |
| 60 | 236 | hPLP1-060 | GGUGUUGUCAGCAUCUUCUdTdT |
| | 237 | hPLP1-060anti | AGAAGAUGCUGACAACACCdTdT |
| 61 | 238 | hPLP1-061 | GUCAAAGCAAGGAUCUUUCdTdT |
| | 239 | hPLP1-061anti | GAAAGAUCCUUGCUUUGACdTdT |
| 62 | 240 | hPLP1-062 | AGAUCUUUGGCGACUACAAdTdT |
| | 241 | hPLP1-062anti | UUGUAGUCGCCAAAGAUCUdTdT |
| 63 | 242 | hPLP1-063 | UGCAGGAGAAGAGGACAAAdTdT |
| | 243 | hPLP1-063anti | UUUGUCCUCUUCUCCUGCAdTdT |
| 64 | 244 | hPLP1-064 | UGGUGGCUAAUGGUGUAACdTdT |
| | 245 | hPLP1-064anti | GUUACACCAUUAGCCACCAdTdT |
| 65 | 246 | hPLP1-065 | AGCUAAUUGAGACCUAUUUdTdT |
| | 247 | hPLP1-065anti | AAAUAGGUCUCAAUUAGCUdTdT |
| 66 | 248 | hPLP1-066 | GUGCUGAUGCCAGAAUGUAdTdT |
| | 249 | hPLP1-066anti | UACAUUCUGGCAUCAGCACdTdT |
| 67 | 250 | hPLP1-067 | UUUCCAAGGAGCUGAGAAUdTdT |
| | 251 | hPLP1-067anti | AUUCUCAGCUCCUUGGAAAdTdT |
| 68 | 252 | hPLP1-068 | UGUGCCUGUGUACAUUUACdTdT |
| | 253 | hPLP1-068anti | GUAAAUGUACACAGGCACAdTdT |
| 69 | 254 | hPLP1-069 | CAGGUACAGAGAAGGAAUGdTdT |
| | 255 | hPLP1-069anti | CAUUCCUUCUCUGUACCUGdTdT |
| 70 | 256 | hPLP1-070 | GCAUAAGGGAGCGUAGAAUdTdT |
| | 257 | hPLP1-070anti | AUUCUACGCUCCCUUAUGCdTdT |
| 71 | 258 | hPLP1-071 | UCAGGUACAGAGAAGGAAUdTdT |
| | 259 | hPLP1-071anti | AUUCCUUCUCUGUACCUGAdTdT |
| 72 | 260 | hPLP1-072 | GCUGCAGCUACACUGGUUUdTdT |
| | 261 | hPLP1-072anti | AAACCAGUGUAGCUGCAGCdTdT |
| 73 | 262 | hPLP1-073 | UCUCCCAUGGAAUGCUUUCdTdT |
| | 263 | hPLP1-073anti | GAAAGCAUUCCAUGGGAGAdTdT |
| 74 | 264 | hPLP1-074 | UGCCUUCCAGUAUGUCAUCdTdT |
| | 265 | hPLP1-074anti | GAUGACAUACUGGAAGGCAdTdT |
| 75 | 266 | hPLP1-075 | GCUGCCACUUACAACUUUGdTdT |
| | 267 | hPLP1-075anti | CAAAGUUGUAAGUGGCAGCdTdT |
| 76 | 268 | hPLP1-076 | ACUUUGCCGUCCUUAAACUdTdT |
| | 269 | hPLP1-076anti | AGUUUAAGGACGGCAAAGUdTdT |
| 77 | 270 | hPLP1-077 | ACCUUACUUACAGCAUAAGdTdT |
| | 271 | hPLP1-077anti | CUUAUGCUGUAAGUAAGGUdTdT |
| 78 | 272 | hPLP1-078 | UGGACUACUGAAGCCCUAAdTdT |
| | 273 | hPLP1-078anti | UUAGGGCUUCAGUAGUCCAdTdT |
| 79 | 274 | hPLP1-079 | CUUCCAGUAUGUCAUCUAUdTdT |
| | 275 | hPLP1-079anti | AUAGAUGACAUACUGGAAGdTdT |
| 80 | 276 | hPLP1-080 | UCUGGUCUCUCUAUUACCAdTdT |
| | 277 | hPLP1-080anti | UGGUAAUAGAGAGACCAGAdTdT |
| 81 | 278 | hPLP1-081 | GUCCCAAAGACAUGGGCUUdTdT |
| | 279 | hPLP1-081anti | AAGCCCAUGUCUUUGGGACdTdT |

Thereafter, an RNA was extracted from the cells, and PLP1 gene expression was evaluated through a quantitative PCR method (n=3). A SuperPrep Cell Lysis RT Kit for qPCR (TOYOBO CO., LTD.) was used for RNA preparation and cDNA synthesis. For the quantitative PCR method, a Taqman probe for the PLP1 gene (Hs00166914 m1, Applied Biosystems) and that for the S18 gene (Hs99999901_s1, Applied Biosystems) were used. Results of the gene expression were normalized to the house keeping gene S18.

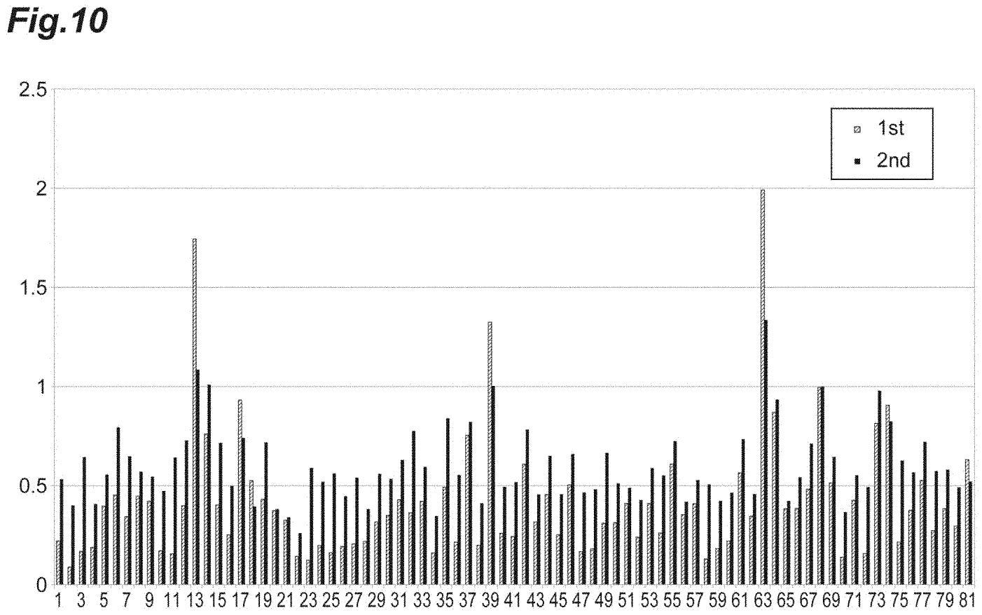

FIG. 10 shows the results. The vertical axis in FIG. 10 represents knockdown efficiencies with reference to U-251MG cells with addition only of transfection solution. Knockdown efficiencies for the PLP1 gene were calculated as relative expression levels assuming the expression level in U-251MG cells with addition only of transfection solution as 1. Selected were 24 pairs of siRNA sequences that lower PLP1 gene expression levels to 50% or less as a mean value both in the first and second trials (Nos. 2, 4, 10, 16, 21, 22, 26, 28, 34, 38, 40, 43, 45, 47, 48, 52, 56, 59, 60, 62, 65, 70, 72, and 80).

### Example 8 Evaluation of Suppression Efficiency of Expression (Knockdown) in Terms of PLP1 Gene Expression Levels—Part 5

On the basis of the 24 pairs of siRNA sequences obtained in Example 7, miRNA sequences were designed (Table 2). Among them, miRNA sequences targeting the sequence encoding the human PLP1 gene were selected (SEQ ID NOs: 2, 29, 30, 31, 32, 33, 35, 36, 42, and 51). Among them, SEQ ID NOs: 2, 29, 31, 32, and 33 are each a sequence directed to a sequence common to mice and primates such as common marmosets.

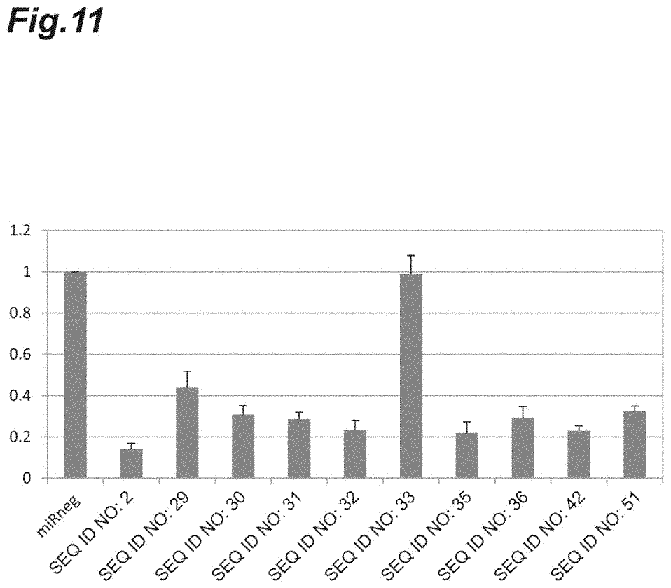

To evaluate the 10 miRNA sequences, each plasmid obtained by cloning any of the sequences into the downstream of a CAG promoter was introduced through a transfection method into HeLa cells overexpressing the coding sequences of the human PLP1 gene. The experimental details of primers used in quantitative PCR for PLP1 gene expression are the same as in Example 6. FIG. 11 shows results of the knockdown. The vertical axis in FIG. 11 represents the PLP1 expression levels±SD relative to the expression when a vector including the miR-neg was added, which was set as 1. FIG. 11 indicates that miRNAs having any of SEQ ID NOs: 2, 29, 30, 31, 32, 35, 36, 42, and 51 knocked down the human PLP1 gene.

### Example 9 Evaluation of Suppression Efficiency of Expression (Knockdown) in Terms of PLP1 Gene Expression Levels—Part 6

Each of the vectors used in Examples 6 and 8, including an miRNA having a nucleotide sequence set forth in any of SEQ ID NOs: 2, 29, 31, 32, and 35, was transfected into U-251MG cells endogenously expressing the human PLP1 gene to evaluate knockdown efficiencies for PLP1 gene expression. A plasmid obtained by cloning into the downstream of a CAG promoter was introduced into cells through a transfection method, and GFP-expressing cells were then exclusively selected by a FACS method. Thereafter, PLP1 gene expression was analyzed though a quantitative PCR method. For the primers, primers having nucleotide sequences set forth in SEQ ID NOs: 280 to 283 were used.

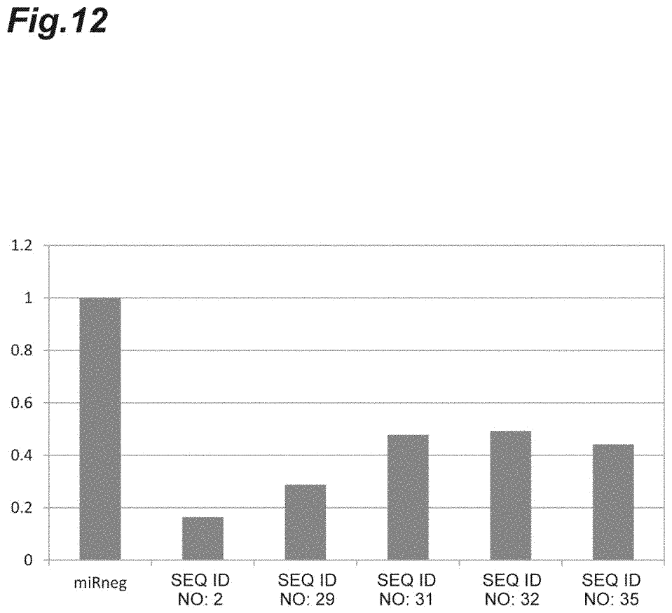

FIG. 12 shows the results. The vertical axis in FIG. 12 represents relative expression levels assuming the PLP1 expression level when a vector including the miR-neg was added as 1. From FIG. 12, vectors including an miRNA having any of SEQ ID NOs: 2, 29, 31, 32, and 35 were found to also have suppressing effect on endogenous PLP1 gene expression.

### Example 10 Evaluation of Suppression Efficiency of Expression (Knockdown) in Terms of PLP1 Gene Expression Levels—Part 7

One microliter of a solution of an scAAV-AAV1/2 vector including the miRNA having SEQ ID NO: 2 from Example 9 (titer: 1.2×10¹² vg/mL) was injected into one striatum, the internal capsule, and the cerebellum of each 10-day-old, wild-type mouse Jc1:B6C3F1 (n=3 per group). The injection method was the same as in Example 2. Thereafter, expression of PLP1 protein in Venus-positive cells was examined through immunostaining to evaluate PLP1 miRNA knockdown efficiencies. Immunostaining was performed with the same method as in Example 2 except that an anti-PLP1 rabbit polyclonal antibody (Numata Y et al., J Biol Chem. 2013 Mar. 15; 288 (11): 7451-66) was used as a primary antibody, and an anti-rabbit fluorescent antibody (Thermo Fisher Scientific, USA) was used as a secondary antibody. Each site of the callosum, striatum, and internal capsule were photographed with a KEYENCE fluorescence microscope (KEYENCE CORPORATION, Japan).

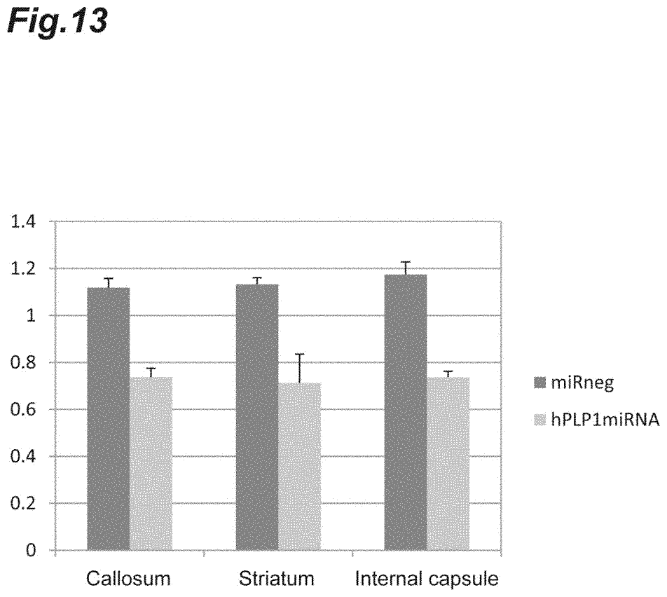

Mean fluorescence intensities of immunostained PLP1 were quantified by using Image J 1.45s (Wayne Rasband, National Institutes of Health, USA), and FIG. 13 shows quantitative analysis of fluorescence intensity through PLP1 immunofluorescence staining. The vertical axis in FIG. 13 represents relative PLP1 expression levels in the infected side with reference to the non-infected side. It can be seen from FIG. 13 that there was almost no difference in expression of PLP1 protein between the side without injection of the miR-neg vector (non-infected side) and the side with vector injection (infected side), whereas for the PLP1 miRNA vector there was 40% of expression-suppressing effect on PLP1 protein in the infected side as compared with the non-infected side.

### INDUSTRIAL APPLICABILITY

The promoter of the present invention and the AAV vector of the present invention can serve as a therapeutic drug for PMD caused by PLP1 duplication. In addition, they can serve as a therapeutic drug for PMD caused by PLP1 point mutation. Furthermore, since PLP1 is one of target antigens for multiple sclerosis, it is expected that they can serve as a therapeutic drug for multiple sclerosis.

1. An oligodendrocyte-specific promoter comprising a nucleic acid having a sequence identity of at least 90% to a nucleotide sequence set forth in SEQ ID NO: 1.

2. The promoter according to claim 1, comprising a nucleic acid having the nucleotide sequence set forth in SEQ ID NO: 1.

3. An oligodendrocyte-specific vector comprising the promoter according to claim 1.

4. The vector according to claim 3, further comprising an miRNA sequence specific to a human PLP1 gene operably linked to the promoter.

5. An miRNA specific to a PLP1 gene, comprising a pair of nucleotide sequences consisting of an antisense sequence and a sense sequence, wherein the pair of nucleotide sequences is selected from the group consisting of pairs of an antisense sequence set forth in a left column of a table below and a sense sequence set forth in a corresponding row of a right column of the table.

| Antisense sequence | Sense sequence |
|---|---|
| AAAGGAAGAAGAAAGAGGCAG (SEQ ID NO: | CTGCCTCTCTTCTTCCTTT (SEQ ID NO: 53) |
| 52) | |
| AACACCAGGAGCCACACAACG (SEQ ID NO: | CGTTGTGTCTCCTGGTGTT (SEQ ID NO: 55) |
| 54) | |
| TTCCATGGGAGAACACCATAC (SEQ ID NO: | GTATGGTGCTCCCATGGAA (SEQ ID NO: 57) |
| 56) | |
| TGAGCAGGGAAACCAGTGTAG (SEQ ID NO: | CTACACTGTTCCCTGCTCA (SEQ ID NO: 59) |
| 58) | |
| AGGGCTTTCTGATTGACAGCC (SEQ ID NO: | GGCTGTCACAGAAAGCCCT (SEQ ID NO: 61) |
| 60) | |
| ACCCCAAAGAAACACAATCCA (SEQ ID NO: | TGGATTGTTTCTTTGGGGT (SEQ ID NO: 63) |
| 62) | |
| ACAAATGCAGCAATAAACAGG (SEQ ID NO: | CCTGTTTAGCTGCATTTGT (SEQ ID NO: 65) |
| 64) | |
| AATAGACTGGCAGGTGGTCCA (SEQ ID NO: | TGGACCACGCCAGTCTATT (SEQ ID NO: 67) |
| 66) | |
| AAAGAATGAGCTTGATGTTGG (SEQ ID NO: | CCAACATCGCTCATTCTTT (SEQ ID NO: 69) |
| 68) | |
| AGATACTCATAGTCTTGGTAG (SEQ ID NO: | CTACCAAGTATGAGTATCT (SEQ ID NO: 71) |
| 70) | |
| AGAAACACAATCCAGTGGCCA (SEQ ID NO: | TGGCCACTATTGTGTTTCT (SEQ ID NO: 73) |
| 72) | |
| AAATAGGTCTCAATTAGCTTT (SEQ ID NO: | AAAGCTAAGAGACCTATTT (SEQ ID NO: 75) |
| 74) | |
| AAGAAACACAATCCAGTGGCC (SEQ ID NO: | GGCCACTGTTGTGTTTCTT (SEQ ID NO: 77) |
| 76) | |
| TAAACAGGTGGAAGGTCATTT (SEQ ID NO: | AAATGACCCCACCTGTTTA (SEQ ID NO: 79) |
| 78) | |
| TTGTAGTCGCCAAAGATCTGC (SEQ ID NO: | GCAGATCTGGCGACTACAA (SEQ ID NO: 81) |
| 80) | |
| AATTAGAGCCTCCATTCCTTT (SEQ ID NO: | AAAGGAATAGGCTCTAATT (SEQ ID NO: 83) |
| 82) | |
| TTAAGGACGGCAAAGTTGTAA (SEQ ID NO: | TTACAACTGCCGTCCTTAA (SEQ ID NO: 85) |
| 84) | |
| TTTAAGGACGGCAAAGTTGTA (SEQ ID NO: | TACAACTTCCGTCCTTAAA (SEQ ID NO: 87) |
| 86) | |
| ATGTCTTTGGGACTCTGACTC (SEQ ID NO: | GAGTCAGACCCAAAGACAT (SEQ ID NO: 89) |
| 88) | |
| TATCTATCCTGTGTCTACCAG (SEQ ID NO: | CTGGTAGACAGGATAGATA (SEQ ID NO: 91) |
| 90) | |
| AAATTACTTTCTGATCCTCAG (SEQ ID NO: | CTGAGGATGAAAGTAATTT (SEQ ID NO: 93) |
| 92) | |
| TCTAACAAGCCCATGTCTTTG (SEQ ID NO: | CAAAGACAGGCTTGTTAGA (SEQ ID NO: 95) |
| 94) | |
| AATTACTTTCTGATCCTCAGG (SEQ ID NO: | CCTGAGGAAGAAAGTAATT (SEQ ID NO: 97) |
| 96) | |
| AGTAAATGTACACAGGCACAG (SEQ ID NO: | CTGTGCCTGTACATTTACT (SEQ ID NO: 99) |
| 98) | |
| TAAGTAAGGTTGGCTGAGTTA (SEQ ID NO: | TAACTCAGAACCTTACTTA (SEQ ID NO: |
| 100) | 101) |
| TTCTGTGGGTGAAAGATCCTT (SEQ ID NO: | AAGGATCTCACCCACAGAA (SEQ ID NO: |
| 102) | 103) |
| AGAAGATGCTGACAACACCCT (SEQ ID NO: | AGGGTGTTCAGCATCTTCT (SEQ ID NO: |
| 104) | 105) |
| AATTGTAGCCGGCTGGCTAGT (SEQ ID NO: | ACTAGCCACGGCTACAATT (SEQ ID NO: |
| 106) | 107) |
| AGATTTGGGCAAACGCTCTTA (SEQ ID NO: | TAAGAGCGTGCCCAAATCT (SEQ ID NO: |
| 108) | 109) |
| ATTCTACGCTCCCTTATGCTG (SEQ ID NO: | CAGCATAAGAGCGTAGAAT (SEQ ID NO: |
| 110) | 111) |
| TGGTAATAGAGAGACCAGAAT (SEQ ID NO: | ATTCTGGTCTCTATTACCA (SEQ ID NO: |
| 112) | 113) |
| TATAGATGGCAAGAGGACCAA (SEQ ID NO: | TTGGTCCTTGCCATCTATA (SEQ ID NO: |
| 114) | 115) |
| AAACCAGTGTAGCTGCAGCCC (SEQ ID NO: | GGGCTGCATACACTGGTTT (SEQ ID NO: |
| 116) | 117) |

6. The miRNA according to claim 5, comprising a nucleotide sequence selected from the group consisting of SEQ ID NOs: 2 to 7 and 24 to 51.

7. The miRNA according to claim 5, comprising a nucleotide sequence selected from the group consisting of SEQ ID NOs: 2 to 5, 7, 24, 26, 29 to 32, 35, 36, 42, and 51.

8. The miRNA according to claim 5, comprising a nucleotide sequence selected from the group consisting of SEQ ID NOs: 2, 79, 31, and 32.

9. A vector comprising a sequence of the miRNA according to claim 5.

10. The vector according to claim 9, wherein the sequence of the miRNA is operably linked to an oligodendrocyte-specific promoter.

11. The vector according to claim 10,
wherein the oligodendrocyte-specific promoter comprises a nucleic acid having a sequence identity, of at least 90% to a nucleotide sequence set forth in SEQ ID NO: 1.

12. The vector according to claim 3, wherein the vector is an adeno-associated virus (AAV) vector.

13-17. (canceled)

18. A method for treating a disease associated with abnormality of a PLP1 gene, the method comprising administering an effective amount of the vector according to claim 3 to a patient in need of treatment.

19. The method according to claim 18, wherein the disease is Pelizaeus-Merzbacher disease, spastic paraplegia type 2, or multiple sclerosis.

20. The method according to claim 18, wherein the disease is Pelizaeus-Merzbacher disease.

21. A method for suppressing expression of a PLP1 gene, the method comprising administering an effective amount of the vector according to claim 3 to a patient in need of treatment.

22-24. (canceled)

25. The vector according to claim 9, wherein the vector is an adeno-associated virus (AAV) vector.

26. A method for treating a disease associated with abnormality of a PLP1 gene, the method comprising administering an effective amount of the vector according to claim 9 to a patient in need of treatment.

27. The method according to claim 26, wherein the disease is Pelizaeus-Merzbacher disease, spastic paraplegia type 2, or multiple sclerosis.

28. The method according to claim 26, wherein the disease is Pelizaeus-Merzbacher disease.

29. A method for suppressing expression of a PLP1 gene, the method comprising administering an effective amount of the vector according to claim 9 to a patient in need of treatment.


SEQUENCE LISTING (283 sequences)

SEQ ID NO: 1 - DNA, 1793 bp, Homo sapiens
ggaaaggtcggggctgagactgactttcggctggcacagggcctggacgaatgaggccagtgaggcatttcagatgcagagtttaccggaatgccagcaaattgcatgataatattttaatccaatgtggtaaaaaaaaaagtttttaattaatgcaaaagtccatgatgaataaaatatcaaaaattaaagacaggatccgactttgcacgaccctgcctcactcacttcctgctaatccacgggaccggggagggtgactgagttacccctcaacacgcagaaaaagctagtggccgctcagctcgcaggcgcagtgggtgggtgcgcgtggggagcaggcgcgcgcagggggcgggaccgggccgaggttcccgcgcgcggcgagagtttcgggttttggttcgcagcgcctgcgcgcagaggagacggcccgcccccagcccgcccgggtgcccgctccgccccccgggctatgtaaagcggccgggctcgggtcgtgccaccgctggactcccgtgtccctccgcgcaggcgggcggccccggagcgctggtgccggcagaggcggcgacggtggcgcccctcctcatcatggtgagaggccgggcggggccgggcacggggtagcaccagggcgggaaacgagtgtcggggcccctcgggaggaagcgtcttgcaaacgaggacccggggctccgggttcgacttccagttttcttggtactcagggcggccctgaggtctgggagaagcggaggggttttggggtgccggagaggccgctgtaaagggaaacggtggtgtttctcaggagggaactcgggaccgcagaagctgctgcgcctctgggagcctgggtgggcctgggccgggagcccgggagctgctcccgaccccacgcaggcccgcgctggggcagccgcttcggcgccccctccccgggcccagtctcatcacgcggaaccgctgccccgcgcccgggtgcgggcgggaggtggagccagttcagaggaggggaccctttacggtaaaagctgccgtcccttgtggcttctagaagataggaaaacaagtggctccgagaggcgcttctgacacacagcacagcggcgggtagagctggatcctaattgcaggtgccctgtgctgccctgttctggacaggcacaaagaccacgactttgtgggggccgggagagggtagaggaggaggtggcaggcttggtgaaagagggcctttgtccagtgctctcctgccctgccccccaccagcccttctgtgggaccattgtacccgcatgccagacaagagagtattatctgttgcggccattgttgggggaagggaggcagtcttggggtaacccttctccactcccccccttctctgtgcactcagccgtggcagctgtggcttggcccagacacagagacccccacctccaaagaggacgtccttagtaggtgccaagcatataagagtgaggccagtcccagaaccgcaggctcctggcgcgccccgcatgcctccagcacgtttacctttccgaagtggcaggaatggggaaagcgcacgcttaggagagcttcagacaagcttccctcttcctcctccacgaccagaagcggaaaggtgctcccggaccgaaagggaaagaaggtccagcactgccccgcttgggaaggcacccacaaccagtctagggactaggggtaaggccggcggggagcccgcgaatgacctgggctgacatctcttcccctccttacacagaacagaggcttctccc

SEQ ID NO: 2 - DNA, 59 bp, Artificial Sequence
  human PLP1-miRNA 1
aaaggaagaagaaagaggcaggttttggccactgactgacctgcctctcttcttccttt

SEQ ID NO: 3 - DNA, 59 bp, Artificial Sequence
  human PLP1 miRNA 2
aacaccaggagccacacaacggttttggccactgactgaccgttgtgtctcctggtgtt

SEQ ID NO: 4 - DNA, 59 bp, Artificial Sequence
  human PLP1 miRNA 3
ttccatgggagaacaccatacgttttggccactgactgacgtatggtgctcccatggaa

SEQ ID NO: 5 - DNA, 59 bp, Artificial Sequence
  human PLP1 miRNA 4
tgagcagggaaaccagtgtaggttttggccactgactgacctacactgttccctgctca

SEQ ID NO: 6 - DNA, 59 bp, Artificial Sequence
  human PLP1 miRNA 5
agggctttctgattgacagccgttttggccactgactgacggctgtcacagaaagccct

SEQ ID NO: 7 - DNA, 59 bp, Artificial Sequence
  human PLP1 miRNA 6
accccaaagaaacacaatccagttttggccactgactgactggattgtttctttggggt

SEQ ID NO: 8 - DNA, 3473 bp, Mus musculus
aatcagaaagcccttttcattgcaggagaagaggacaaagatactcagagagaaaaagtaaaggacagaagaaggagactggagagaccaggatccttccagctgagcaaagtcagccgcaaaacagactagccaacaggctacaattggagtcagagtgccaaagacatgggcttgttagagtgttgtgctagatgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagaatgtatggtgttctcccatggaatgctttccctggcaaggtttgtggctccaaccttctgtccatctgcaaaacagctgagttccaaatgaccttccacctgtttattgctgcgtttgtgggtgctgcggccacactagtttccctgctcaccttcatgattgctgccacttacaacttcgccgtccttaaactcatgggccgaggcaccaagttctgagctcccatagaaactcccctttgtctaatagcaaggctctaaccacacagcctacagtgttgtgttttaactctgcctttgccactgattggccctcttcttacttgatgagtataacaagaaaggagagtcttgcagtgattaatctctctctgtggactctccctcttagtacctcttttagtcattttgctccacagcaggctcctgctagaaatgggggatgcctgagaaggtgactccccagctgcaagtcgcagaggagtgaaagctctaattgattttgcaagcatctcctgaagaccaggatgtgcttccttctcaaagggcacttccaactgaggagagcagaacggaaaggttctcaggtagagagcagaaatgtccctggtcttcttgccatcagtaggagtcaaatacattctctttgatgcacaaaaccaagaactcactcttaccttcctgtttccactgaagacagaagaaaataaaaagaatgctagcagagcaatatagcatttgcccaaatctgcctcctgcagctgggagaagggtgtcaaagcaaggatctttcgcccttagaaagagagctctgacgccagtggcaatggactatttaagccctaactcagccaaccttccttacggcaattagggagcacagtgcctgtatagacaaagcggggcggagggggggggcatcatctgtccttatagctcattaggaagagaaacagtgttgtcaggatcatctcactcccttctccttgataacagctaccatgacaaccttgtggtttccaaggagctgagaatagaaaggaactagcttatttgaaataagactgtgacctaaggagcatcagttggtggatgctaaaggtgtaatttgaaatggccttcgggtaaatgcaagatacttaactctttggatagcatgtgttcttcccccacccctatccgctagttctggcccctggcctctggcataatatcttcacaatggtgctttttttcctggggttttatccattcactcatagcaggtgattagacgatcttgattagtttcatatttcccaattgtttatctcttgtttggagttgtatcagaaagacctggaggatgattctttgagcatagttctttttgaaaacaagaaagagaaactgggcagaaagcatcacaaaaatatttgaaattgtacggtcccatgaaattattgggaattcccccaagtagtctaccatttgtagaactaggcttgataaatttgaacctcaatttgaataattggtctggtattttcttttctaataaatgacagatgattttacttgctaatattatctcagcattttgataatttaggcttaccatagaagttactgtctcttggtatatataggtcacataatagattctgccagctgttagctgttcagttcataagcttccatagagctctggagccgcagagaggacaggcagaatttgaaacctaaagaactcccagatttcaggcttatcctgtatttgttaactttgggtgaaagaaagaaagaaagaaagaaagaaagaaagaaagaaagaaagaaagaaagaaagaaagaaagaaagaaagaaagaaagaaagaaagaaaaagaaaggaaggaaggaaggaaggaagaaagaaagaaagaaagaaagaaagaaagaaagaaagaaagaaagaaagaaagaaagaaagagaaaaaaaaagcccctgatcgaatttcctggaggaaaagttattgtagctgtttcattgtagatttgtgctgtcattccccaaagtgctttctgctgtgttgaaagagatataagaatttacaagaagacacttgagacttgttcttgggccaatatataaggtaaacaagcaggatgcacaagagtgaggagagctaaaaggacatgtaagaaaccaatcaagatcaaggaaggtgaaataatctatatcttttattttgttttggtttaatataacagataaccaaccattcccttaaaaatctcacatgcacacacacacacacacacacacacacgtacaaagagagttaatcaactgcaagtgtttccttcatttctgatagagaattttgattttaacaacataaaggataaacttttagaaactcatcttacaaaatgtattttataaaattaaagaaaataaaattaagaatgttctcaatcaaacatcgtgtcctttgagtgaattgttctatttgacctcaataacaggtacttaattatagttagctcgaggtgctcatgtatctttcaggccatgtaagttattcttatactacttctatgaaaaatgtaatagataatgcattattattattattgtttcttttttatactaaagatatgaaaaaatatatgcaaaatgcaaaacaattaccgaaagaaactcagtaaatacttgtctcaaattga

SEQ ID NO: 9 - DNA, 59 bp, Artificial Sequence
  mouse PLP1-miRNA
actccaaagaaacacaatccagttttggccactgactgactggattgtttctttggagt

SEQ ID NO: 10 - DNA, 59 bp, Artificial Sequence
  mouse miR-neg
gtatgcatcgaatgagattccgttttggccactgactgacggaatctctcgatgcatac

SEQ ID NO: 11 - DNA, 27 bp, Artificial Sequence
  5′ flanking sequence
ctggaggcttgctgaaggctgtatgct

SEQ ID NO: 12 - DNA, 45 bp, Artificial Sequence
  3′ flanking sequence
caggacacaaggcctgttactagcactcacatggaacaaatggcc

SEQ ID NO: 13 - DNA, 5538 bp, Artificial Sequence
  pW-CAG-Venus-WPRE
gcggccgcacgcgagcttgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgcccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcccaggcggcccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaaggccaggccggccggatcctctagagtcgacctgcagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgaggcaagcttgctctcgagagatctacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcgtaggtaaccacgtgcggaccgagcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagcttgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcccatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaga

SEQ ID NO: 14 - DNA, 733 bp, Artificial Sequence
  Venus
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaaggccaggccggcc

SEQ ID NO: 15 - DNA, 5155 bp, Artificial Sequence
  pscW-PABPN1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatgcccaatccatgggcctgcaggcagctgcgcgctcgctcgctcactgaggccgcccgggcaaagcccgggcgtcgggcgacctttggtcgcccggcctcagtgagcgagcgagcgcgcagagagggagtggggttatcggcgcgccgcggccgctctagaaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccggatccggggccggcctggccaggcctcgtacgatcgatcctaggatatcacgcgtcttaagtcagtaggggctgtaccagctggtggcccgggcccggccccggtacacccggccccggggccggctgttgaagccgctgtagaaccggctccggctgctgttgtagttggtggtccgggcccggtaccgggcccgggggaagccccggtcggtggtgctgatgccgggccggttggtccgcttggggatcaccttgatctgccggccccggaacaggctctcgtccagggccaggctggtccgcacgctctccttgtcgctgaactcgatgtaggcgaagcccttggggtggccgctgaacttgtcgcacaggatggtcacccggttcacgctgccgcagccgtggaagtgggcctccagctcctcggcggtggcgccgtagtccacgttgcccacgtagatgctccgggcgtcggcctccatcttctcctcgatgctcatgatcacggggccggcgttgccggggggggggctcatgttcatctgcttctccacctcgttctgcagctccttcagcttctcggcctcctcctccatctcccgcacccgggccttgatggcctccagctcggggtcctcgatggcgccgtcgccggggtcgccctccaccaggccgggctcctcctcctcctcctggctgccgggggcgccgctgccggggccggggccgggggcgccggggggggcccggggccgggggggctcctcctcgggctcgggctcgggctcgggctccagcagcagctcctcgggctccagctcctcgctctccaggccgttgccgtagtcgccggcgccgccgggggcgccctcgccggcctcgccgccggcgccgggcaccaggtgccgccgccggccggggccgctgccccggccgccggcggcgcccgctgctgctgccgccgccgccgccgccgctgctgctgctgctgctgctgccatggtgggccgcctgggccgaattcgtcgactgcagaattcgaagcttgagctcgagatctgagtccggtagcgctagcttttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcactagtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcctaggatttgggatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 5865 bp, Artificial Sequence
  Pscw-hCNP-Venus-PLP1 miRNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatgcccaatccatgggcctgcaggcagctgcgcgctcgctcgctcactgaggccgcccgggcaaagcccgggcgtcgggcgacctttggtcgcccggcctcagtgagcgagcgagcgcgcagagagggagtggggttatcggcgcgccgcggccgcacgcgagcttgtcgacattgattattgactagttaatgcaaaagtccatgatgaataaaatatcaaaaattaaagacaggatccgactttgcacgaccctgcctcactcacttcctgctaatccacgggaccggggagggtgactgagttacccctcaacacgcagaaaaagctagcggccgctcagctcgcaggcgcagtgggtgggtgcgcgtggggagcaggcgcgcgcagggggcgggaccgggccgaggttcccgcgcgcggcgagagtttcgggttttggttcgcagcgcctgcgcgcagaggagacggcccgcccccagcccgcccgggtgcccgctccgccccccgggctatgtaaagcggccgggctcgggttgtgccaccgctggactcccgtgtccctccgcgcaggcgggcggccccggagcgctggtgccggcagaggcggcgacggtggcgcccctcctcatcatggtgagaggccgggcggggccgggcacggggtagcaccagggcgggaaacgagtgtcggggcccctcgggaggaagcgtcttgcaaacgaggacccggggctccgggttcgacttccagttttcttggtactcagggcggccctgaggtctgggagaagcggaggggttttggggtgccggagaggccgctgtaaagggaaacggtggtgtttctcaggagggaactcgggaccgcagaagctgctgcgcctctgggagcctgggtgggcctgggccgggagcccgggagctgctcccgaccccacgcaggcccgcgctggggcagccgcttcggcgccccctccccgggcccagtctcatcacgcggaaccgctgccccgcgcccgggtgcgggcgggaggtggagccagttcagaggaggggaccctttacggtaaaagctgccgtcccttgtggcttctagaagataggaaaacaagtggctccgagaggcgcttctgacacacagcacagcggcgggtagagctggatcctaattgcaggtgccctgtgctgccctgttctggacaggcacaaagaccacgactttgtgggggccgggagagggtagaggaggaggtggcaggcttggtgaaagagggcctttgtccagtgctctcctgccctgccccccaccagcccttctgtgggaccattgtacccgcatgccagacaagagagtattatctgttgcggccattgttgggggaagggaggcagtcttggggtaacccttctccactcccccccttctctgtgcactcagccgtggcagctgtggcttggcccagacacagagacccccacctccaaagaggacgtccttagtaggtgccaagcatataagagtgaggccagtcccagaaccgcaggctcctggcgcgccccgcatgcctccagcacgtttacctttccgaagtggcaggaatggggaaagcgcacgcttaggagagcttcagacaagcttccctcttcctcctccacgaccagaagcggaaaggtgctcccggaccgaaagggaaagaaggtccagcactgccccgcttgggaaggcacccacaaccagtctagggactaggggtaaggccggcggggagcccgcgaatgacctgggctgacatctcttcccctccttacacagaacagaggcttctcccgaattcggcccaggcggcccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaaggccaggccggccggatcctggaggcttgctgaaggctgtatgctgactccaaagaaacacaatccagttttggccactgactgactggattgtttctttggagtcaggacacaaggcctgttactagcactcacatggaacaaatggcccagatctggccgcactcgaggcaagcttgctctcgagagatctacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcctaggatttgggatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 5865 bp, Artificial Sequence
  Pscw-hCNP-Venus-miRneg
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatgcccaatccatgggcctgcaggcagctgcgcgctcgctcgctcactgaggccgcccgggcaaagcccgggcgtcgggcgacctttggtcgcccggcctcagtgagcgagcgagcgcgcagagagggagtggggttatcggcgcgccgcggccgcacgcgagcttgtcgacattgattattgactagttaatgcaaaagtccatgatgaataaaatatcaaaaattaaagacaggatccgactttgcacgaccctgcctcactcacttcctgctaatccacgggaccggggagggtgactgagttacccctcaacacgcagaaaaagctagcggccgctcagctcgcaggcgcagtgggtgggtgcgcgtggggagcaggcgcgcgcagggggcgggaccgggccgaggttcccgcgcgcggcgagagtttcgggttttggttcgcagcgcctgcgcgcagaggagacggcccgcccccagcccgcccgggtgcccgctccgccccccgggctatgtaaagcggccgggctcgggttgtgccaccgctggactcccgtgtccctccgcgcaggcgggcggccccggagcgctggtgccggcagaggcggcgacggtggcgcccctcctcatcatggtgagaggccgggcggggccgggcacggggtagcaccagggcgggaaacgagtgtcggggcccctcgggaggaagcgtcttgcaaacgaggacccggggctccgggttcgacttccagttttcttggtactcagggcggccctgaggtctgggagaagcggaggggttttggggtgccggagaggccgctgtaaagggaaacggtggtgtttctcaggagggaactcgggaccgcagaagctgctgcgcctctgggagcctgggtgggcctgggccgggagcccgggagctgctcccgaccccacgcaggcccgcgctggggcagccgcttcggcgccccctccccgggcccagtctcatcacgcggaaccgctgccccgcgcccgggtgcgggcgggaggtggagccagttcagaggaggggaccctttacggtaaaagctgccgtcccttgtggcttctagaagataggaaaacaagtggctccgagaggcgcttctgacacacagcacagcggcgggtagagctggatcctaattgcaggtgccctgtgctgccctgttctggacaggcacaaagaccacgactttgtgggggccgggagagggtagaggaggaggtggcaggcttggtgaaagagggcctttgtccagtgctctcctgccctgccccccaccagcccttctgtgggaccattgtacccgcatgccagacaagagagtattatctgttgcggccattgttgggggaagggaggcagtcttggggtaacccttctccactcccccccttctctgtgcactcagccgtggcagctgtggcttggcccagacacagagacccccacctccaaagaggacgtccttagtaggtgccaagcatataagagtgaggccagtcccagaaccgcaggctcctggcgcgccccgcatgcctccagcacgtttacctttccgaagtggcaggaatggggaaagcgcacgcttaggagagcttcagacaagcttccctcttcctcctccacgaccagaagcggaaaggtgctcccggaccgaaagggaaagaaggtccagcactgccccgcttgggaaggcacccacaaccagtctagggactaggggtaaggccggcggggagcccgcgaatgacctgggctgacatctcttcccctccttacacagaacagaggcttctcccgaattcggcccaggcggcccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaaggccaggccggccggatcctggaggcttgctgaaggctgtatgctggtatgcatcgaatgagattccgttttggccactgactgacggaatctctcgatgcataccaggacacaaggcctgttactagcactcacatggaacaaatggcccagatctggccgcactcgaggcaagcttgctctcgagagatctacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcctaggatttgggatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - DNA, 24 bp, Artificial Sequence
  PLP1 forward primer
gttccagaggccaacatcaagctc

SEQ ID NO: 19 - DNA, 24 bp, Artificial Sequence
  PLP1 reverse primer
agccatacaacagtcagggcatag

SEQ ID NO: 20 - DNA, 19 bp, Artificial Sequence
  Olig2 forward primer
gggaggtcatgccttacgc

SEQ ID NO: 21 - DNA, 19 bp, Artificial Sequence
  Olig2 reverse primer
ctccagcgagttggtgagc

SEQ ID NO: 22 - DNA, 22 bp, Artificial Sequence
  actin forward primer
cacagcttctttgcagctcctt

SEQ ID NO: 23 - DNA, 19 bp, Artificial Sequence
  actin reverse primer
gacgaccagcgcagcgata

SEQ ID NO: 24 - DNA, 59 bp, Artificial Sequence
  human PLP1 miRNA 7
acaaatgcagcaataaacagggttttggccactgactgaccctgtttagctgcatttgt

SEQ ID NO: 25 - DNA, 59 bp, Artificial Sequence
  human PLP1 miRNA 8
aatagactggcaggtggtccagttttggccactgactgactggaccacgccagtctatt

SEQ ID NO: 26 - DNA, 59 bp, Artificial Sequence
  human PLP1 miRNA 9
aaagaatgagcttgatgttgggttttggccactgactgacccaacatcgctcattcttt

SEQ ID NO: 27 - DNA, 59 bp, Artificial Sequence
  human PLP1 miRNA 10
agatactcatagtcttggtaggttttggccactgactgacctaccaagtatgagtatct

SEQ ID NO: 28 - DNA, 59 bp, Artificial Sequence
  human PLP1 miRNA 11
aagcccatgtctttgggactcgttttggccactgactgacgagtcccagacatgggctt

SEQ ID NO: 29 - DNA, 59 bp, Artificial Sequence
  pre-miRNA
agaaacacaatccagtggccagttttggccactgactgactggccactattgtgtttct

SEQ ID NO: 30 - DNA, 59 bp, Artificial Sequence
  pre-miRNA
aaataggtctcaattagctttgttttggccactgactgacaaagctaagagacctattt

SEQ ID NO: 31 - DNA, 59 bp, Artificial Sequence
  pre-miRNA
aagaaacacaatccagtggccgttttggccactgactgacggccactgttgtgtttctt

SEQ ID NO: 32 - DNA, 59 bp, Artificial Sequence
  pre-miRNA
taaacaggtggaaggtcatttgttttggccactgactgacaaatgaccccacctgttta

SEQ ID NO: 33 - DNA, 59 bp, Artificial Sequence
  pre-miRNA
ttgtagtcgccaaagatctgcgttttggccactgactgacgcagatctggcgactacaa

SEQ ID NO: 34 - DNA, 59 bp, Artificial Sequence
  pre-miRNA
aattagagcctccattcctttgttttggccactgactgacaaaggaataggctctaatt

SEQ ID NO: 35 - DNA, 59 bp, Artificial Sequence
  pre-miRNA
ttaaggacggcaaagttgtaagttttggccactgactgacttacaactgccgtccttaa

SEQ ID NO: 36 - DNA, 59 bp, Artificial Sequence
  pre-miRNA
tttaaggacggcaaagttgtagttttggccactgactgactacaacttccgtccttaaa

SEQ ID NO: 37 - DNA, 59 bp, Artificial Sequence
  pre-miRNA
atgtctttgggactctgactcgttttggccactgactgacgagtcagacccaaagacat

SEQ ID NO: 38 - DNA, 59 bp, Artificial Sequence
  pre-miRNA
tatctatcctgtgtctaccaggttttggccactgactgacctggtagacaggatagata

SEQ ID NO: 39 - DNA, 59 bp, Artificial Sequence
  pre-miRNA
aaattactttctgatcctcaggttttggccactgactgacctgaggatgaaagtaattt

SEQ ID NO: 40 - DNA, 59 bp, Artificial Sequence
  pre-miRNA
tctaacaagcccatgtctttggttttggccactgactgaccaaagacaggcttgttaga

SEQ ID NO: 41 - DNA, 59 bp, Artificial Sequence
  pre-miRNA
aattactttctgatcctcagggttttggccactgactgaccctgaggaagaaagtaatt

SEQ ID NO: 42 - DNA, 59 bp, Artificial Sequence
  pre-miRNA
agtaaatgtacacaggcacaggttttggccactgactgacctgtgcctgtacatttact

SEQ ID NO: 43 - DNA, 59 bp, Artificial Sequence
  pre-miRNA
taagtaaggttggctgagttagttttggccactgactgactaactcagaaccttactta

SEQ ID NO: 44 - DNA, 59 bp, Artificial Sequence
  pre-miRNA
ttctgtgggtgaaagatccttgttttggccactgactgacaaggatctcacccacagaa

SEQ ID NO: 45 - DNA, 59 bp, Artificial Sequence
  pre-miRNA
agaagatgctgacaacaccctgttttggccactgactgacagggtgttcagcatcttct

SEQ ID NO: 46 - DNA, 59 bp, Artificial Sequence
  pre-miRNA
aattgtagccggctggctagtgttttggccactgactgacactagccacggctacaatt

SEQ ID NO: 47 - DNA, 59 bp, Artificial Sequence
  pre-miRNA
agatttgggcaaacgctcttagttttggccactgactgactaagagcgtgcccaaatct

SEQ ID NO: 48 - DNA, 59 bp, Artificial Sequence
  pre-miRNA
attctacgctcccttatgctggttttggccactgactgaccagcataagagcgtagaat

SEQ ID NO: 49 - DNA, 59 bp, Artificial Sequence
  pre-miRNA
tggtaatagagagaccagaatgttttggccactgactgacattctggtctctattacca

SEQ ID NO: 50 - DNA, 59 bp, Artificial Sequence
  pre-miRNA
tatagatggcaagaggaccaagttttggccactgactgacttggtccttgccatctata

SEQ ID NO: 51 - DNA, 59 bp, Artificial Sequence
  pre-miRNA
aaaccagtgtagctgcagcccgttttggccactgactgacgggctgcatacactggttt

SEQ ID NO: 52 - DNA, 21 bp, Artificial Sequence
  human PLP1 antisense sequence
aaaggaagaagaaagaggcag

SEQ ID NO: 53 - DNA, 19 bp, Artificial Sequence
  human PLP1 sense sequence
ctgcctctcttcttccttt

SEQ ID NO: 54 - DNA, 21 bp, Artificial Sequence
  human PLP1 antisense sequence
aacaccaggagccacacaacg

SEQ ID NO: 55 - DNA, 19 bp, Artificial Sequence
  human PLP1 sense sequence
cgttgtgtctcctggtgtt

SEQ ID NO: 56 - DNA, 21 bp, Artificial Sequence
  human PLP1 antisense sequence
ttccatgggagaacaccatac

SEQ ID NO: 57 - DNA, 19 bp, Artificial Sequence
  human PLP1 sense sequence
gtatggtgctcccatggaa

SEQ ID NO: 58 - DNA, 21 bp, Artificial Sequence
  human PLP1 antisense sequence
tgagcagggaaaccagtgtag

SEQ ID NO: 59 - DNA, 19 bp, Artificial Sequence
  human PLP1 sense sequence
ctacactgttccctgctca

SEQ ID NO: 60 - DNA, 21 bp, Artificial Sequence
  human PLP1 antisense sequence
agggctttctgattgacagcc

SEQ ID NO: 61 - DNA, 19 bp, Artificial Sequence
  human PLP1 sense sequence
ggctgtcacagaaagccct

SEQ ID NO: 62 - DNA, 21 bp, Artificial Sequence
  human PLP1 antisense sequence
accccaaagaaacacaatcca

SEQ ID NO: 63 - DNA, 19 bp, Artificial Sequence
  human PLP1 sense sequence
tggattgtttctttggggt

SEQ ID NO: 64 - DNA, 21 bp, Artificial Sequence
  human PLP1 antisense sequence
acaaatgcagcaataaacagg

SEQ ID NO: 65 - DNA, 19 bp, Artificial Sequence
  human PLP1 sense sequence
cctgtttagctgcatttgt

SEQ ID NO: 66 - DNA, 21 bp, Artificial Sequence
  human PLP1 antisense sequence
aatagactggcaggtggtcca

SEQ ID NO: 67 - DNA, 19 bp, Artificial Sequence
  human PLP1 sense sequence
tggaccacgccagtctatt

SEQ ID NO: 68 - DNA, 21 bp, Artificial Sequence
  human PLP1 antisense sequence
aaagaatgagcttgatgttgg

SEQ ID NO: 69 - DNA, 19 bp, Artificial Sequence
  human PLP1 sense sequence
ccaacatcgctcattcttt

SEQ ID NO: 70 - DNA, 21 bp, Artificial Sequence
  human PLP1 antisense sequence
agatactcatagtcttggtag

SEQ ID NO: 71 - DNA, 19 bp, Artificial Sequence
  human PLP1 sense sequence
ctaccaagtatgagtatct

SEQ ID NO: 72 - DNA, 21 bp, Artificial Sequence
  human PLP1 antisense sequence
agaaacacaatccagtggcca

SEQ ID NO: 73 - DNA, 19 bp, Artificial Sequence
  human PLP1 sense sequence
tggccactattgtgtttct

SEQ ID NO: 74 - DNA, 21 bp, Artificial Sequence
  human PLP1 antisense sequence
aaataggtctcaattagcttt

SEQ ID NO: 75 - DNA, 19 bp, Artificial Sequence
  human PLP1 sense sequence
aaagctaagagacctattt

SEQ ID NO: 76 - DNA, 21 bp, Artificial Sequence
  human PLP1 antisense sequence
aagaaacacaatccagtggcc

SEQ ID NO: 77 - DNA, 19 bp, Artificial Sequence
  human PLP1 sense sequence
ggccactgttgtgtttctt

SEQ ID NO: 78 - DNA, 21 bp, Artificial Sequence
  human PLP1 antisense sequence
taaacaggtggaaggtcattt

SEQ ID NO: 79 - DNA, 19 bp, Artificial Sequence
  human PLP1 sense sequence
aaatgaccccacctgttta

SEQ ID NO: 80 - DNA, 21 bp, Artificial Sequence
  human PLP1 antisense sequence
ttgtagtcgccaaagatctgc

SEQ ID NO: 81 - DNA, 19 bp, Artificial Sequence
  human PLP1 sense sequence
gcagatctggcgactacaa

SEQ ID NO: 82 - DNA, 21 bp, Artificial Sequence
  human PLP1 antisense sequence
aattagagcctccattccttt

SEQ ID NO: 83 - DNA, 19 bp, Artificial Sequence
  human PLP1 sense sequence
aaaggaataggctctaatt

SEQ ID NO: 84 - DNA, 21 bp, Artificial Sequence
  human PLP1 antisense sequence
ttaaggacggcaaagttgtaa

SEQ ID NO: 85 - DNA, 19 bp, Artificial Sequence
  human PLP1 sense sequence
ttacaactgccgtccttaa

SEQ ID NO: 86 - DNA, 21 bp, Artificial Sequence
  human PLP1 antisense sequence
tttaaggacggcaaagttgta

SEQ ID NO: 87 - DNA, 19 bp, Artificial Sequence
  human PLP1 sense sequence
tacaacttccgtccttaaa

SEQ ID NO: 88 - DNA, 21 bp, Artificial Sequence
  human PLP1 antisense sequence
atgtctttgggactctgactc

SEQ ID NO: 89 - DNA, 19 bp, Artificial Sequence
  human PLP1 sense sequence
gagtcagacccaaagacat

SEQ ID NO: 90 - DNA, 21 bp, Artificial Sequence
  human PLP1 antisense sequence
tatctatcctgtgtctaccag

SEQ ID NO: 91 - DNA, 19 bp, Artificial Sequence
  human PLP1 sense sequence
ctggtagacaggatagata

SEQ ID NO: 92 - DNA, 21 bp, Artificial Sequence
  human PLP1 antisense sequence
aaattactttctgatcctcag

SEQ ID NO: 93 - DNA, 19 bp, Artificial Sequence
  human PLP1 sense sequence
ctgaggatgaaagtaattt

SEQ ID NO: 94 - DNA, 21 bp, Artificial Sequence
  human PLP1 antisense sequence
tctaacaagcccatgtctttg

SEQ ID NO: 95 - DNA, 19 bp, Artificial Sequence
  human PLP1 sense sequence
caaagacaggcttgttaga

SEQ ID NO: 96 - DNA, 21 bp, Artificial Sequence
  human PLP1 antisense sequence
aattactttctgatcctcagg

SEQ ID NO: 97 - DNA, 19 bp, Artificial Sequence
  human PLP1 sense sequence
cctgaggaagaaagtaatt

SEQ ID NO: 98 - DNA, 21 bp, Artificial Sequence
  human PLP1 antisense sequence
agtaaatgtacacaggcacag

SEQ ID NO: 99 - DNA, 19 bp, Artificial Sequence
  human PLP1 sense sequence
ctgtgcctgtacatttact

SEQ ID NO: 100 - DNA, 21 bp, Artificial Sequence
  human PLP1 antisense sequence
taagtaaggttggctgagtta

SEQ ID NO: 101 - DNA, 19 bp, Artificial Sequence
  human PLP1 sense sequence
taactcagaaccttactta

SEQ ID NO: 102 - DNA, 21 bp, Artificial Sequence
  human PLP1 antisense sequence
ttctgtgggtgaaagatcctt

SEQ ID NO: 103 - DNA, 19 bp, Artificial Sequence
  human PLP1 sense sequence
aaggatctcacccacagaa

SEQ ID NO: 104 - DNA, 21 bp, Artificial Sequence
  human PLP1 antisense sequence
agaagatgctgacaacaccct

SEQ ID NO: 105 - DNA, 19 bp, Artificial Sequence
  human PLP1 sense sequence
agggtgttcagcatcttct

SEQ ID NO: 106 - DNA, 21 bp, Artificial Sequence
  human PLP1 antisense sequence
aattgtagccggctggctagt

SEQ ID NO: 107 - DNA, 19 bp, Artificial Sequence
  human PLP1 sense sequence
actagccacggctacaatt

SEQ ID NO: 108 - DNA, 21 bp, Artificial Sequence
  human PLP1 antisense sequence
agatttgggcaaacgctctta

SEQ ID NO: 109 - DNA, 19 bp, Artificial Sequence
  human PLP1 sense sequence
taagagcgtgcccaaatct

SEQ ID NO: 110 - DNA, 21 bp, Artificial Sequence
  human PLP1 antisense sequence
attctacgctcccttatgctg

SEQ ID NO: 111 - DNA, 19 bp, Artificial Sequence
  human PLP1 sense sequence
cagcataagagcgtagaat

SEQ ID NO: 112 - DNA, 21 bp, Artificial Sequence
  human PLP1 antisense sequence
tggtaatagagagaccagaat

SEQ ID NO: 113 - DNA, 19 bp, Artificial Sequence
  human PLP1 sense sequence
attctggtctctattacca

SEQ ID NO: 114 - DNA, 21 bp, Artificial Sequence
  human PLP1 antisense sequence
tatagatggcaagaggaccaa

SEQ ID NO: 115 - DNA, 19 bp, Artificial Sequence
  human PLP1 sense sequence
ttggtccttgccatctata

SEQ ID NO: 116 - DNA, 21 bp, Artificial Sequence
  human PLP1 antisense sequence
aaaccagtgtagctgcagccc

SEQ ID NO: 117 - DNA, 19 bp, Artificial Sequence
  human PLP1 sense sequence
gggctgcatacactggttt

SEQ ID NO: 118 - DNA, 23 bp, Artificial Sequence
  hPLP1-001
ugaccuuccaccuguuuaudtdt

SEQ ID NO: 119 - DNA, 23 bp, Artificial Sequence
  hPLP1-001anti
auaaacagguggaaggucadtdt

SEQ ID NO: 120 - DNA, 23 bp, Artificial Sequence
  hPLP1-002
gugccuguguacauuuacudtdt

SEQ ID NO: 121 - DNA, 23 bp, Artificial Sequence
  hPLP1-002anti
aguaaauguacacaggcacdtdt

SEQ ID NO: 122 - DNA, 23 bp, Artificial Sequence
  hPLP1-003
uugcccaaaucugccuauudtdt

SEQ ID NO: 123 - DNA, 23 bp, Artificial Sequence
  hPLP1-003anti
aauaggcagauuugggcaadtdt

SEQ ID NO: 124 - DNA, 23 bp, Artificial Sequence
  hPLP1-004
aggaauggaggcucuaauudtdt

SEQ ID NO: 125 - DNA, 23 bp, Artificial Sequence
  hPLP1-004anti
aauuagagccuccauuccudtdt

SEQ ID NO: 126 - DNA, 23 bp, Artificial Sequence
  hPLP1-005
uggccacuggauuguguuudtdt

SEQ ID NO: 127 - DNA, 23 bp, Artificial Sequence
  hPLP1-005anti
aaacacaauccaguggccadtdt

SEQ ID NO: 128 - DNA, 23 bp, Artificial Sequence
  hPLP1-006
gcgggugugucauuguuugdtdt

SEQ ID NO: 129 - DNA, 23 bp, Artificial Sequence
  hPLP1-006anti
caaacaaugacacacccgcdtdt

SEQ ID NO: 130 - DNA, 23 bp, Artificial Sequence
  hPLP1-007
gaccuuccaccuguuuauudtdt

SEQ ID NO: 131 - DNA, 23 bp, Artificial Sequence
  hPLP1-007anti
aauaaacagguggaaggucdtdt

SEQ ID NO: 132 - DNA, 23 bp, Artificial Sequence
  hPLP1-008
acucccuucuccuugauaadtdt

SEQ ID NO: 133 - DNA, 23 bp, Artificial Sequence
  hPLP1-008anti
uuaucaaggagaagggagudtdt

SEQ ID NO: 134 - DNA, 23 bp, Artificial Sequence
  hPLP1-009
cugcaagucacaaaggaaudtdt

SEQ ID NO: 135 - DNA, 23 bp, Artificial Sequence
  hPLP1-009anti
auuccuuugugacuugcagdtdt

SEQ ID NO: 136 - DNA, 23 bp, Artificial Sequence
  hPLP1-010
gguccucuugccaucuauadtdt

SEQ ID NO: 137 - DNA, 23 bp, Artificial Sequence
  hPLP1-010anti
uauagauggcaagaggaccdtdt

SEQ ID NO: 138 - DNA, 23 bp, Artificial Sequence
  hPLP1-011
ccucguuagggaagagaaadtdt

SEQ ID NO: 139 - DNA, 23 bp, Artificial Sequence
  hPLP1-011anti
uuucucuucccuaacgaggdtdt

SEQ ID NO: 140 - DNA, 23 bp, Artificial Sequence
  hPLP1-012
ggagaagaggacaaagauadtdt

SEQ ID NO: 141 - DNA, 23 bp, Artificial Sequence
  hPLP1-012anti
uaucuuuguccucuucuccdtdt

SEQ ID NO: 142 - DNA, 23 bp, Artificial Sequence
  hPLP1-013
ggccaacaucaagcucauudtdt

SEQ ID NO: 143 - DNA, 23 bp, Artificial Sequence
  hPLP1-013anti
aaugagcuugauguuggccdtdt

SEQ ID NO: 144 - DNA, 23 bp, Artificial Sequence
  hPLP1-014
aggccaacaucaagcucaudtdt

SEQ ID NO: 145 - DNA, 23 bp, Artificial Sequence
  hPLP1-014anti
augagcuugauguuggccudtdt

SEQ ID NO: 146 - DNA, 23 bp, Artificial Sequence
  hPLP1-015
cucccuucuccuugauaacdtdt

SEQ ID NO: 147 - DNA, 23 bp, Artificial Sequence
  hPLP1-015anti
guuaucaaggagaagggagdtdt

SEQ ID NO: 148 - DNA, 23 bp, Artificial Sequence
  hPLP1-016
gccucuuucuucuuccuuudtdt

SEQ ID NO: 149 - DNA, 23 bp, Artificial Sequence
  hPLP1-016anti
aaaggaagaagaaagaggcdtdt

SEQ ID NO: 150 - DNA, 23 bp, Artificial Sequence
  hPLP1-017
cugugccuguguacauuuadtdt

SEQ ID NO: 151 - DNA, 23 bp, Artificial Sequence
  hPLP1-017anti
uaaauguacacaggcacagdtdt

SEQ ID NO: 152 - DNA, 23 bp, Artificial Sequence
  hPLP1-018
aaggaauggaggcucuaaudtdt

SEQ ID NO: 153 - DNA, 23 bp, Artificial Sequence
  hPLP1-018anti
auuagagccuccauuccuudtdt

SEQ ID NO: 154 - DNA, 23 bp, Artificial Sequence
  hPLP1-019
aggguguugucagcaucuudtdt

SEQ ID NO: 155 - DNA, 23 bp, Artificial Sequence
  hPLP1-019anti
aagaugcugacaacacccudtdt

SEQ ID NO: 156 - DNA, 23 bp, Artificial Sequence
  hPLP1-020
acagcugaguuccaaaugadtdt

SEQ ID NO: 157 - DNA, 23 bp, Artificial Sequence
  hPLP1-020anti
ucauuuggaacucagcugudtdt

SEQ ID NO: 158 - DNA, 23 bp, Artificial Sequence
  hPLP1-021
gccacuggauuguguuucudtdt

SEQ ID NO: 159 - DNA, 23 bp, Artificial Sequence
  hPLP1-021anti
agaaacacaauccaguggcdtdt

SEQ ID NO: 160 - DNA, 23 bp, Artificial Sequence
  hPLP1-022
acaacuuugccguccuuaadtdt

SEQ ID NO: 161 - DNA, 23 bp, Artificial Sequence
  hPLP1-022anti
uuaaggacggcaaaguugudtdt

SEQ ID NO: 162 - DNA, 23 bp, Artificial Sequence
  hPLP1-023
gcugaugccagaauguaugdtdt

SEQ ID NO: 163 - DNA, 23 bp, Artificial Sequence
  hPLP1-023anti
cauacauucuggcaucagcdtdt

SEQ ID NO: 164 - DNA, 23 bp, Artificial Sequence
  hPLP1-024
uugccguccuuaaacucaudtdt

SEQ ID NO: 165 - DNA, 23 bp, Artificial Sequence
  hPLP1-024anti
augaguuuaaggacggcaadtdt

SEQ ID NO: 166 - DNA, 23 bp, Artificial Sequence
  hPLP1-025
cugccucuuucuucuuccudtdt

SEQ ID NO: 167 - DNA, 23 bp, Artificial Sequence
  hPLP1-025anti
aggaagaagaaagaggcagdtdt

SEQ ID NO: 168 - DNA, 23 bp, Artificial Sequence
  hPLP1-026
ccacuggauuguguuucuudtdt

SEQ ID NO: 169 - DNA, 23 bp, Artificial Sequence
  hPLP1-026anti
aagaaacacaauccaguggdtdt

SEQ ID NO: 170 - DNA, 23 bp, Artificial Sequence
  hPLP1-027
ggaucagaaaguaauuucudtdt

SEQ ID NO: 171 - DNA, 23 bp, Artificial Sequence
  hPLP1-027anti
agaaauuacuuucugauccdtdt

SEQ ID NO: 172 - DNA, 23 bp, Artificial Sequence
  hPLP1-028
uagccagccggcuacaauudtdt

SEQ ID NO: 173 - DNA, 23 bp, Artificial Sequence
  hPLP1-028anti
aauuguagccggcuggcuadtdt

SEQ ID NO: 174 - DNA, 23 bp, Artificial Sequence
  hPLP1-029
agcguagaaucuguguagadtdt

SEQ ID NO: 175 - DNA, 23 bp, Artificial Sequence
  hPLP1-029anti
ucuacacagauucuacgcudtdt

SEQ ID NO: 176 - DNA, 23 bp, Artificial Sequence
  hPLP1-030
ucccuucuccuugauaacadtdt

SEQ ID NO: 177 - DNA, 23 bp, Artificial Sequence
  hPLP1-030anti
uguuaucaaggagaagggadtdt

SEQ ID NO: 178 - DNA, 23 bp, Artificial Sequence
  hPLP1-031
uuccaaggagcugagaauadtdt

SEQ ID NO: 179 - DNA, 23 bp, Artificial Sequence
  hPLP1-031anti
uauucucagcuccuuggaadtdt

SEQ ID NO: 180 - DNA, 23 bp, Artificial Sequence
  hPLP1-032
ugccagaauguaugguguudtdt

SEQ ID NO: 181 - DNA, 23 bp, Artificial Sequence
  hPLP1-032anti
aacaccauacauucuggcadtdt

SEQ ID NO: 182 - DNA, 23 bp, Artificial Sequence
  hPLP1-033
augccuuccaguaugucaudtdt

SEQ ID NO: 183 - DNA, 23 bp, Artificial Sequence
  hPLP1-033anti
augacauacuggaaggcaudtdt

SEQ ID NO: 184 - DNA, 23 bp, Artificial Sequence
  hPLP1-034
caacuuugccguccuuaaadtdt

SEQ ID NO: 185 - DNA, 23 bp, Artificial Sequence
  hPLP1-034anti
uuuaaggacggcaaaguugdtdt

SEQ ID NO: 186 - DNA, 23 bp, Artificial Sequence
  hPLP1-035
ccaacaucaagcucauucudtdt

SEQ ID NO: 187 - DNA, 23 bp, Artificial Sequence
  hPLP1-035anti
agaaugagcuugauguuggdtdt

SEQ ID NO: 188 - DNA, 23 bp, Artificial Sequence
  hPLP1-036
uggccuuacaccucguuagdtdt

SEQ ID NO: 189 - DNA, 23 bp, Artificial Sequence
  hPLP1-036anti
cuaacgagguguaaggccadtdt

SEQ ID NO: 190 - DNA, 23 bp, Artificial Sequence
  hPLP1-037
aggacaucccgacaaguuudtdt

SEQ ID NO: 191 - DNA, 23 bp, Artificial Sequence
  hPLP1-037anti
aaacuugucgggauguccudtdt

SEQ ID NO: 192 - DNA, 23 bp, Artificial Sequence
  hPLP1-038
gucagagucccaaagacaudtdt

SEQ ID NO: 193 - DNA, 23 bp, Artificial Sequence
  hPLP1-038anti
augucuuugggacucugacdtdt

SEQ ID NO: 194 - DNA, 23 bp, Artificial Sequence
  hPLP1-039
uugcaggagaagaggacaadtdt

SEQ ID NO: 195 - DNA, 23 bp, Artificial Sequence
  hPLP1-039anti
uuguccucuucuccugcaadtdt

SEQ ID NO: 196 - DNA, 23 bp, Artificial Sequence
  hPLP1-040
gguagacacaggauagauadtdt

SEQ ID NO: 197 - DNA, 23 bp, Artificial Sequence
  hPLP1-040anti
uaucuauccugugucuaccdtdt

SEQ ID NO: 198 - DNA, 23 bp, Artificial Sequence
  hPLP1-041
ucagccaaccuuacuuacadtdt

SEQ ID NO: 199 - DNA, 23 bp, Artificial Sequence
  hPLP1-041anti
uguaaguaagguuggcugadtdt

SEQ ID NO: 200 - DNA, 23 bp, Artificial Sequence
  hPLP1-042
auggaacugccucuuucuudtdt

SEQ ID NO: 201 - DNA, 23 bp, Artificial Sequence
  hPLP1-042anti
aagaaagaggcaguuccaudtdt

SEQ ID NO: 202 - DNA, 23 bp, Artificial Sequence
  hPLP1-043
acucagccaaccuuacuuadtdt

SEQ ID NO: 203 - DNA, 23 bp, Artificial Sequence
  hPLP1-043anti
uaaguaagguuggcugagudtdt

SEQ ID NO: 204 - DNA, 23 bp, Artificial Sequence
  hPLP1-044
cuuccacugauggaaacaadtdt

SEQ ID NO: 205 - DNA, 23 bp, Artificial Sequence
  hPLP1-044anti
uuguuuccaucaguggaagdtdt

SEQ ID NO: 206 - DNA, 23 bp, Artificial Sequence
  hPLP1-045
augaccuuccaccuguuuadtdt

SEQ ID NO: 207 - DNA, 23 bp, Artificial Sequence
  hPLP1-045anti
uaaacagguggaaggucaudtdt

SEQ ID NO: 208 - DNA, 23 bp, Artificial Sequence
  hPLP1-046
ucuccugaggaucagaaagdtdt

SEQ ID NO: 209 - DNA, 23 bp, Artificial Sequence
  hPLP1-046anti
cuuucugauccucaggagadtdt

SEQ ID NO: 210 - DNA, 23 bp, Artificial Sequence
  hPLP1-047
gaggaucagaaaguaauuudtdt

SEQ ID NO: 211 - DNA, 23 bp, Artificial Sequence
  hPLP1-047anti
aaauuacuuucugauccucdtdt

SEQ ID NO: 212 - DNA, 23 bp, Artificial Sequence
  hPLP1-048
ggaucuuucacccacagaadtdt

SEQ ID NO: 213 - DNA, 23 bp, Artificial Sequence
  hPLP1-048anti
uucugugggugaaagauccdtdt

SEQ ID NO: 214 - DNA, 23 bp, Artificial Sequence
  hPLP1-049
cugaggaucagaaaguaaudtdt

SEQ ID NO: 215 - DNA, 23 bp, Artificial Sequence
  hPLP1-049anti
auuacuuucugauccucagdtdt

SEQ ID NO: 216 - DNA, 23 bp, Artificial Sequence
  hPLP1-050
cugguagacacaggauagadtdt

SEQ ID NO: 217 - DNA, 23 bp, Artificial Sequence
  hPLP1-050anti
ucuauccugugucuaccagdtdt

SEQ ID NO: 218 - DNA, 23 bp, Artificial Sequence
  hPLP1-051
gcaguugcugguggcuaaudtdt

SEQ ID NO: 219 - DNA, 23 bp, Artificial Sequence
  hPLP1-051anti
auuagccaccagcaacugcdtdt

SEQ ID NO: 220 - DNA, 23 bp, Artificial Sequence
  hPLP1-052
aagacaugggcuuguuagadtdt

SEQ ID NO: 221 - DNA, 23 bp, Artificial Sequence
  hPLP1-052anti
ucuaacaagcccaugucuudtdt

SEQ ID NO: 222 - DNA, 23 bp, Artificial Sequence
  hPLP1-053
uugcugccacuuacaacuudtdt

SEQ ID NO: 223 - DNA, 23 bp, Artificial Sequence
  hPLP1-053anti
aaguuguaaguggcagcaadtdt

SEQ ID NO: 224 - DNA, 23 bp, Artificial Sequence
  hPLP1-054
ucguuagggaagagaaacadtdt

SEQ ID NO: 225 - DNA, 23 bp, Artificial Sequence
  hPLP1-054anti
uguuucucuucccuaacgadtdt

SEQ ID NO: 226 - DNA, 23 bp, Artificial Sequence
  hPLP1-055
uccacugauggaaacaaagdtdt

SEQ ID NO: 227 - DNA, 23 bp, Artificial Sequence
  hPLP1-055anti
cuuuguuuccaucaguggadtdt

SEQ ID NO: 228 - DNA, 23 bp, Artificial Sequence
  hPLP1-056
agagcguuugcccaaaucudtdt

SEQ ID NO: 229 - DNA, 23 bp, Artificial Sequence
  hPLP1-056anti
agauuugggcaaacgcucudtdt

SEQ ID NO: 230 - DNA, 23 bp, Artificial Sequence
  hPLP1-057
ugauugcugccacuuacaadtdt

SEQ ID NO: 231 - DNA, 23 bp, Artificial Sequence
  hPLP1-057anti
uuguaaguggcagcaaucadtdt

SEQ ID NO: 232 - DNA, 23 bp, Artificial Sequence
  hPLP1-058
ggagcguagaaucuguguadtdt

SEQ ID NO: 233 - DNA, 23 bp, Artificial Sequence
  hPLP1-058anti
uacacagauucuacgcuccdtdt

SEQ ID NO: 234 - DNA, 22 bp, Artificial Sequence
  hPLP1-059
ugaggaucagaaaguaauudtd

SEQ ID NO: 235 - DNA, 23 bp, Artificial Sequence
  hPLP1-059anti
aauuacuuucugauccucadtdt

SEQ ID NO: 236 - DNA, 23 bp, Artificial Sequence
  hPLP1-060
gguguugucagcaucuucudtdt

SEQ ID NO: 237 - DNA, 23 bp, Artificial Sequence
  hPLP1-060anti
agaagaugcugacaacaccdtdt

SEQ ID NO: 238 - DNA, 23 bp, Artificial Sequence
  hPLP1-061
gucaaagcaaggaucuuucdtdt

SEQ ID NO: 239 - DNA, 23 bp, Artificial Sequence
  hPLP1-061anti
gaaagauccuugcuuugacdtdt

SEQ ID NO: 240 - DNA, 23 bp, Artificial Sequence
  hPLP1-062
agaucuuuggcgacuacaadtdt

SEQ ID NO: 241 - DNA, 23 bp, Artificial Sequence
  hPLP1-062anti
uuguagucgccaaagaucudtdt

SEQ ID NO: 242 - DNA, 23 bp, Artificial Sequence
  hPLP1-063
ugcaggagaagaggacaaadtdt

SEQ ID NO: 243 - DNA, 23 bp, Artificial Sequence
  hPLP1-063anti
uuuguccucuucuccugcadtdt

SEQ ID NO: 244 - DNA, 23 bp, Artificial Sequence
  hPLP1-064
ugguggcuaaugguguaacdtdt

SEQ ID NO: 245 - DNA, 23 bp, Artificial Sequence
  hPLP1-064anti
guuacaccauuagccaccadtdt

SEQ ID NO: 246 - DNA, 23 bp, Artificial Sequence
  hPLP1-065
agcuaauugagaccuauuudtdt

SEQ ID NO: 247 - DNA, 23 bp, Artificial Sequence
  hPLP1-065anti
aaauaggucucaauuagcudtdt

SEQ ID NO: 248 - DNA, 23 bp, Artificial Sequence
  hPLP1-066
gugcugaugccagaauguadtdt

SEQ ID NO: 249 - DNA, 23 bp, Artificial Sequence
  hPLP1-066anti
uacauucuggcaucagcacdtdt

SEQ ID NO: 250 - DNA, 23 bp, Artificial Sequence
  hPLP1-067
uuuccaaggagcugagaaudtdt

SEQ ID NO: 251 - DNA, 23 bp, Artificial Sequence
  hPLP1-067anti
auucucagcuccuuggaaadtdt

SEQ ID NO: 252 - DNA, 23 bp, Artificial Sequence
  hPLP1-068
ugugccuguguacauuuacdtdt

SEQ ID NO: 253 - DNA, 23 bp, Artificial Sequence
  hPLP1-068anti
guaaauguacacaggcacadtdt

SEQ ID NO: 254 - DNA, 23 bp, Artificial Sequence
  hPLP1-069
cagguacagagaaggaaugdtdt

SEQ ID NO: 255 - DNA, 23 bp, Artificial Sequence
  hPLP1-069anti
cauuccuucucuguaccugdtdt

SEQ ID NO: 256 - DNA, 23 bp, Artificial Sequence
  hPLP1-070
gcauaagggagcguagaaudtdt

SEQ ID NO: 257 - DNA, 23 bp, Artificial Sequence
  hPLP1-070anti
auucuacgcucccuuaugcdtdt

SEQ ID NO: 258 - DNA, 23 bp, Artificial Sequence
  hPLP1-071
ucagguacagagaaggaaudtdt

SEQ ID NO: 259 - DNA, 23 bp, Artificial Sequence
  hPLP1-071anti
auuccuucucuguaccugadtdt

SEQ ID NO: 260 - DNA, 23 bp, Artificial Sequence
  hPLP1-072
gcugcagcuacacugguuudtdt

SEQ ID NO: 261 - DNA, 23 bp, Artificial Sequence
  hPLP1-072anti
aaaccaguguagcugcagcdtdt

SEQ ID NO: 262 - DNA, 23 bp, Artificial Sequence
  hPLP1-073
ucucccauggaaugcuuucdtdt

SEQ ID NO: 263 - DNA, 23 bp, Artificial Sequence
  hPLP1-073anti
gaaagcauuccaugggagadtdt

SEQ ID NO: 264 - DNA, 23 bp, Artificial Sequence
  hPLP1-074
ugccuuccaguaugucaucdtdt

SEQ ID NO: 265 - DNA, 23 bp, Artificial Sequence
  hPLP1-074anti
gaugacauacuggaaggcadtdt

SEQ ID NO: 266 - DNA, 23 bp, Artificial Sequence
  hPLP1-075
gcugccacuuacaacuuugdtdt

SEQ ID NO: 267 - DNA, 23 bp, Artificial Sequence
  hPLP1-075anti
caaaguuguaaguggcagcdtdt

SEQ ID NO: 268 - DNA, 23 bp, Artificial Sequence
  hPLP1-076
acuuugccguccuuaaacudtdt

SEQ ID NO: 269 - DNA, 23 bp, Artificial Sequence
  hPLP1-076anti
aguuuaaggacggcaaagudtdt

SEQ ID NO: 270 - DNA, 23 bp, Artificial Sequence
  hPLP1-077
accuuacuuacagcauaagdtdt

SEQ ID NO: 271 - DNA, 23 bp, Artificial Sequence
  hPLP1-077anti
cuuaugcuguaaguaaggudtdt

SEQ ID NO: 272 - DNA, 23 bp, Artificial Sequence
  hPLP1-078
uggacuacugaagcccuaadtdt

SEQ ID NO: 273 - DNA, 23 bp, Artificial Sequence
  hPLP1-078anti
uuagggcuucaguaguccadtdt

SEQ ID NO: 274 - DNA, 23 bp, Artificial Sequence
  hPLP1-079
cuuccaguaugucaucuaudtdt

SEQ ID NO: 275 - DNA, 23 bp, Artificial Sequence
  hPLP1-079anti
auagaugacauacuggaagdtdt

SEQ ID NO: 276 - DNA, 23 bp, Artificial Sequence
  hPLP1-080
ucuggucucucuauuaccadtdt

SEQ ID NO: 277 - DNA, 23 bp, Artificial Sequence
  hPLP1-080anti
ugguaauagagagaccagadtdt

SEQ ID NO: 278 - DNA, 23 bp, Artificial Sequence
  hPLP1-081
gucccaaagacaugggcuudtdt

SEQ ID NO: 279 - DNA, 23 bp, Artificial Sequence
  hPLP1-081anti
aagcccaugucuuugggacdtdt

SEQ ID NO: 280 - DNA, 21 bp, Artificial Sequence
  human PLP1 forward primer
gctccaaccttctgtccatct

SEQ ID NO: 281 - DNA, 20 bp, Artificial Sequence
  human PLP1 reverse primer
acggcaaagttgtaagtggc

SEQ ID NO: 282 - DNA, 24 bp, Artificial Sequence
  human beta-action forward primer
gacaggatgcagaaggagattact

SEQ ID NO: 283 - DNA, 22 bp, Artificial Sequence
  human beta-actin reverse primer
tgatccacatctgctggaaggt